(19) United States
(12) Patent Application Publication
(10) Pub. No.: US 2006/0035946 A1
(43) Pub. Date: Feb. 16, 2006

(54) Amyloid-binding, metal-chelating agents

(75) Inventors: Xudong Huang, Andover, MA (US); Jonathan L. Kremsky, Arlington, MA (US); Perry L. Catchings SR., Boston, MA (US)

Correspondence Address: CHOATE, HALL & STEWART LLP, TWO INTERNATIONAL PLACE, BOSTON, MA, 02110, US

(73) Assignees: The General Hospital Corporation; Prime Organics, Inc.

(21) Appl. No.: 11/096,919

(22) Filed: Apr. 1, 2005

Related U.S. Application Data
(63) Continuation-in-part of application No. 10/762,965, filed on Jan. 22, 2004.
(60) Provisional application No. 60/441,719, filed on Jan. 22, 2003.

(51) Int. Cl. A61K 31/428; C07D 417/02

(52) U.S. Cl. 514/367; 548/185

(57) ABSTRACT

The present invention relates to the diagnosis, prevention, and treatment of pathophysiological conditions associated with amyloid accumulation. Bifunctional therapeutic molecules and contrast imaging agents exhibiting a high affinity for amyloid deposits, and pharmaceutical compositions thereof are described. The invention also provides methods of using these bifunctional molecules, contrast imaging agents, and pharmaceutical compositions for detecting the presence of amyloid deposits using imaging techniques; and for preventing or treating amyloid-related conditions, such as, for example, Alzheimer's disease.

### RELATED APPLICATION

This application claims priority to co-pending Application U.S. Ser. No. 10/762,965 filed Jan. 22, 2004, which in turn claims priority to Provisional Patent Application No. 60/441,719, filed Jan. 22, 2003, each of which is incorporated herein by reference in its entirety.

### GOVERNMENT INTERESTS

The work described herein was funded by the National Institutes of Health/National Institute of Mental Health (Grant No. 5K01 MH002001-O₂). The United States government may have certain rights in the invention.

### BACKGROUND OF THE INVENTION

Amyloidosis is a group of diseases and disorders characterized by the accumulation of a protein-like substance, called amyloid, in one or more organs and tissues of the body. Amyloid accumulation, which may happen systemically or locally, can impair normal vital functions and cause organ failure. At least 15 types of amyloidosis (each one associated with deposits of a different kind of amyloid protein) have been identified. The nature of the accumulated protein as well as the location of the buildup determine the symptoms, which may vary from mild to life threatening. Amyloid deposits are an important part of the pathology of clinical conditions such as Alzheimer's disease, adult-onset diabetes, chronic inflammatory diseases, dialysis-associated arthropathy, tumors, and familial neuropathies.

In amyloidosis, abnormal folding and polymerization of usually soluble and functional proteins into an insoluble β-sheet-rich quaternary structure causes the aggregated proteins to be excreted from the cells and form extracellular amyloid deposits (i.e., fibrils, filaments, plaques, and tangles). Among the 20 proteins that can undergo such transformation, some accumulate preferentially in the brain and are associated with neurodegenerative conditions. These proteins include, for example, the prion protein, which is associated with the Prion diseases; and the amyloid-β peptide (Aβ), deposits of which are found in the brains of patients with Alzheimer's disease, Down's syndrome, Lewy body dementia, hereditary cerebral hemorrhage with amyloidosis (Dutch type), Guam Parkinson-Dementia, and head trauma.

Although amyloidoses are generally rare pathophysiological conditions, Alzheimer's disease (AD) is the most prevalent form of dementia in the United States. Epidemiological studies have estimated that 40 to 50% of all people in their late 80's have AD (D. A. Evans et al., JAMA, 1989, 262: 2551-2556; R. Katzman, Neurology, 1993, 43: 13-20). The first symptom of the disease is usually memory deficits, followed by impairments in language, cognition, and mobility, and ultimately a loss of mental function so debilitating that the patients become entirely dependent on other people for their everyday care. The deterioration, which is irreversible, eventually leads to death. In addition to the enormous impact it has on individuals and families, AD also presents a major public health problem. According to the National Institute on Aging, an estimated 4 million Americans currently have the disease and approximately 360,000 new cases are diagnosed each year (R. Brookmeyer et al., Am. J. Public. Health. 1998, 88: 1337-1342). With annual national direct and indirect costs of caring for AD patients estimated to be as much as $100 billion (R. L. Ernst et al., Arch. Neurol. 1997, 54: 687-693), AD also puts a heavy economic burden on society.

In Alzheimer's disease patients, aggregation of the amyloid-β peptide leads to the formation of amyloid deposits in the cerebral vasculature and senile plaques in the neocortex (C. L. Masters et al., Proc. Natl. Acad. Sci. USA, 1985, 82: 4245-4249). Because the diagnosis is difficult to establish without invasive biopsy and is only unequivocally achieved through post-mortem examination of brain tissue (G. McKhann et al., Neurology, 1984, 34: 939-944; D. M. Mann, Mech. Ageing Dev. 1985, 31: 213-255), studying AD remains a daunting task, and, so far, the development of treatments and therapies has been elusive. Various FDA-approved therapeutics that act as “cognitive enhancers” (e.g., Aricept®, Cognex®, Exelon®, and Mentane®) provide slight relief in some AD patients. However, these agents do not alter the underlying course of the disease, and, currently, there is no cure or effective treatment for Alzheimer's disease at any stage of its development.

Amyloid accumulation is consistently found to be most concentrated in regions of high neuronal cell death. This correlation is supported by the fact that the amyloid-β peptide acquires high neuronal toxicity when aggregated in a specific β-sheet conformation (J. Y. Koh et al., Brain Res. 1990; 533: 315-320; B. A. Yankner et al., Science, 1990, 250: 279-282). Although growing evidence suggests that amyloid deposits are intimately associated with the neuronal dysfunction seen in AD patients (J. W. Kelly, Proc. Natl. Acad. Sci. USA, 1998, 95: 930-932), the mechanism of toxicity and the neurochemical events that cause Aβ deposition are still unclear.

Transition metals have recently been postulated to play a critical role in the pathogenesis of Alzheimer's disease (C. S. Atwood et al., Met. Ions Biol. Syst., 1999, 36: 309-364; A. I. Bush, Curr. Opin. Chem. Biol. 2000, 4: 184-191). In mice and humans, iron and copper levels have been shown to increase with normal aging in several tissues including the brain (H. R. Massie et al., Mech. Ageing Dev. 1979, 10: 93-99; B. Drayer et al., Am. J. Roentgenol. 1986, 147: 103-110; G. Bartzokis et al., Magn. Reson. Imaging 1997, 15: 29-35; L. Del Corso et al., Panminerva Med. 2000, 42: 273-277). Transition metal imbalances are also observed in the brains of AD patients (J. R. Connor et al., J. Neurosci. Res. 1992, 31: 327-335; D. A. Loeffler et al., Brain Res. 1996, 738: 265-274; M. A. Deibel et al., J. Neurol. Sci. 1996, 143: 137-142; R. Cornett et al., Neurotox. 1998, 19: 339-345), where zinc, copper, and iron are found concentrated in and around amyloid plaques (M. A. Lovell et al., J. Neurol. Sci. 1998, 158: 47-52). Furthermore, the amyloid-β peptide exhibits a high affinity for transition metal ions; and the binding of Zn²⁺ and, to a lesser extent, of Cu²⁺ and Fe³⁺ to Aβ markedly increases its aggregation and the formation of amyloid deposits (A. I. Bush et al., Science, 1994, 265: 1464-1467). Both processes (aggregation and deposition) can be reversed in the presence of metal-chelating agents (X. Huang et al., J. Biol. Chem. 1997, 272: 26464-26470; C. S. Atwood et al., J. Biol. Chem. 1998, 273: 12817-12826; R. A. Chemy et al., J. Biol. Chem. 1999, 274: 23223-23228).

In addition to promoting protein aggregation and amyloid accumulation, the binding of redox active transition metals (i.e., Cu²⁺ and Fe³⁺) to Aβ also leads to the generation of reactive oxygen species (X. Huang et al., J. Biol. Chem. 1999, 274: 37111-37116; X. Huang et al., Biochem. 1999, 38: 7609-7616; L. M. Sayre et al., J. Neurochem. 2000, 74: 270-279), which are known to have deleterious effects on a wide variety of biomolecules. Biometal- and amyloid-mediated production of reactive oxygen species is believed to be responsible, at least in part, for the oxidative stress observed in the brains of AD patients (M. A. Pappolla et al., Am. J. Pathol. 1992; 40: 621-628; W. R. Markesbery, Free Radic. Biol. Med. 1997, 23:134-147; P. Gabbita et al., J. Neurochem. 1998, 71: 2034-2040; M. A. Smith et al., Antioxid. Redox Signal, 2000, 2: 413-420; M. P. Cuajungco et al., J. Biol. Chem. 2000, 275: 19439-19422).

The discovery that transition metal ions may play a role in some of the pathological effects of Alzheimer's disease has provided a new route for the development of diagnostic methods and therapeutic treatments. For example, in U.S. Pat. No. 6,323,218, metal chelators or metal-complexing compounds (such as EDTA, bathophenanthroline, bathocuproine, and penicillamine) are described as therapeutics for the treatment of pathophysiological conditions associated with amyloidosis. Recently, Clioquinol, an orally bioavailable metal chelator, has been shown to induce a marked inhibition of cortical amyloid accumulation in the Tg2576 transgenic mouse model for Alzheimer's disease (R. A. Chemy et al., Neuron. 2001, 30: 665-676). Although the results of these studies are promising and suggest that metal chelators may be of therapeutic value for the treatment of conditions associated with amyloid accumulation, potential side effects of many non-specific metal chelators may prove too great for clinical use as they may perturb the normal physiological function of other metal-requiring biomolecules.

Therefore, the development of effective therapeutic agents and methods for the early diagnosis, prevention, and treatment of amyloidoses in general and of Alzheimer's disease in particular, remains highly desirable.

### SUMMARY OF THE INVENTION

The present invention relates to the diagnosis, prevention, and treatment of pathophysiological conditions associated with amyloid accumulation. In particular, the invention encompasses reagents and strategies for detecting the presence of amyloid deposits, and for preventing or treating amyloid-related conditions. In certain preferred embodiments, the invention allows the diagnosis, prevention, and treatment of pathophysiological conditions associated with aggregation and accumulation of amyloid and amyloid-like proteins in the brain.

In one aspect, the invention provides targeted therapeutic reagents that act as metal chelators and show some degree of attraction for amyloid deposits. More specifically, the present invention provides bifunctional molecules comprising at least one metal-chelating moiety associated with at least one amyloid-binding moiety. Preferably, the amyloid-binding moiety exhibits high affinity and specificity for Aβ amyloid deposits. In certain preferred embodiments, the amyloid-binding moiety is blood-brain barrier permeable. For example, in certain such embodiments, the amyloid-binding moiety may be a benzothiazole derivative. In other preferred embodiments, the metal-chelating moiety binds with high affinity transition metal ions that are biologically relevant, such as zinc II (Zn²⁺), copper II (Cu²⁺), and iron III (Fe³⁺). For example, in such embodiments, the metal-chelating moiety may be DTPA or an α-lipoic acid derivative.

Preferred bifunctional molecules of the invention include compound XH1 and its analogues, the chemical structures of which are presented in FIG. 4. Other preferred bifunctional molecules of the invention include compound XH2 and its analogues, the chemical structures of which are presented in FIG. 6.

In another aspect, the invention provides targeted reagents that show some degree of attraction for amyloid deposits, and are detectable by imaging techniques. More specifically, the invention provides contrast imaging agents comprising at least one imaging moiety associated with at least one amyloid-binding moiety. In certain preferred embodiments, the amyloid-binding moiety is blood-brain barrier permeable. Preferably, the amyloid-binding moiety exhibits high affinity and specificity for Aβ amyloid deposits. For example, preferred amyloid-binding moieties may be benzothiazole derivatives. The imaging moiety may be any suitable entity known in the art to be detectable by imaging techniques. In certain preferred embodiments, the imaging moiety comprises at least one metal-chelating moiety complexed to a detectable metal entity. Preferably, the metal-chelating moiety is complexed to a physiologically acceptable metal entity. In preferred embodiments, the metal entity is a paramagnetic metal ion and the contrast imaging agent is detectable by Magnetic Resonance Imaging (MRI). Preferably, the paramagnetic metal ion is gadolinium III (Gd³⁺). In other preferred embodiments, the metal entity is a radionuclide and the contrast imaging agent is detectable by Single Photon Emission Computed Tomography (SPECT). Preferably, the radionuclide is technetium-99m (^{99m}Tc).

The present invention also provides contrast imaging agents comprising at least one metal-chelating moiety associated with at least one amyloid-binding moiety labeled with a stable paramagnetic isotope that is detectable by Nuclear Magnetic Resonance (NMR). In preferred embodiments, the stable paramagnetic isotope is carbon-13 (¹³C) or fluorine-19 (¹⁹F); and the contrast imaging agent is detectable by Magnetic Resonance Spectroscopy (MRS).

Preferred inventive contrast imaging agents are gadolinium III (Gd³⁺) complexes of the bifunctional molecules described herein.

In another aspect, the invention provides pharmaceutical compositions. The inventive pharmaceutical compositions comprise at least one reagent of the invention, or a physiologically tolerable salt thereof, and at least one pharmaceutically acceptable carrier. In these pharmaceutical compositions, the reagent is present in an amount sufficient to fulfill its intended purpose. More specifically, the present invention provides pharmaceutical compositions comprising an effective amount of at least one bifunctional molecule, or a physiologically tolerable salt thereof, and at least one pharmaceutically acceptable carrier. Also provided are pharmaceutical compositions comprising an imaging effective amount of at least one contrast imaging agent, or a physiologically tolerable salt thereof, and at least one pharmaceutically acceptable carrier. In preferred embodiments, the imaging moiety in the contrast imaging agent comprises at least one metal-chelating moiety complexed to gadolinium III (Gd³⁺) or to technetium-99m (^{99m}Tc). In other preferred embodiments, the amyloid-binding moiety in the contrast imaging agent is labeled with carbon-13 (¹³C) or fluorine-19 (¹⁹F).

In yet another aspect, the invention provides methods for reducing or inhibiting amyloid toxicity in vitro or in vivo. In certain preferred embodiments, the invention allows for reduction or inhibition of amyloid toxicity by preventing, slowing down, or stopping amyloid accumulation; and/or by promoting, inducing, or otherwise facilitating dissolution of amyloid deposits. In other preferred embodiments, the invention allows for reduction or inhibition of amyloid toxicity by decreasing, inhibiting, or otherwise interfering with amyloid-mediated production of reactive oxygen species.

More specifically, methods are provided for reducing or inhibiting amyloid toxicity in a system, comprising contacting the system with a bifunctional molecule of the invention, or a pharmaceutical composition thereof. The system may be any biological entity known to be able to produce and/or contain amyloid deposits. For example, the system may be a cell, a biological fluid, a biological tissue or an animal. The system may originate from a live patient (e.g., it may be obtained by biopsy) or a deceased patient (e.g., it may be obtained at autopsy). The patient may be a human or another mammal. In preferred embodiments, the cell, biological fluid, or biological tissue originates from a patient suspected of having a pathophysiological condition associated with amyloid accumulation.

Also provided herein are methods for treating a patient with a pathophysiological condition associated with amyloid accumulation, comprising administering to the patient an effective amount of a bifunctional molecule of the invention, or a pharmaceutical composition thereof. In certain preferred embodiments, the pathophysiological condition is associated with accumulation of the amyloid-β peptide.

In yet another aspect, the invention provides methods for detecting the presence of amyloid deposits in a system or in a patient. In preferred embodiments, the inventive methods are based on the use of targeted contrast imaging agents and imaging techniques.

More specifically, the invention provides methods for detecting the presence of amyloid deposits in a system comprising contacting the system with an imaging effective amount of a contrast imaging agent, or a pharmaceutical composition thereof. The contacting is preferably carried out under conditions that allow the contrast imaging agent to interact with an amyloid deposit present in the system so that the interaction results in the binding of the contrast imaging agent to the amyloid deposit. The contrast imaging agent bound to amyloid deposits present in the system is then detected using an imaging technique and one or more images of at least part of the system are generated. In certain preferred embodiments, the inventive method is used for identifying potential therapeutic agents for the treatment of pathophysiological conditions associated with amyloid accumulation. The invention includes the therapeutic agents identified by this method.

The present invention also provides methods for detecting the presence of amyloid deposits in a patient. These methods comprise administering to the patient an imaging effective amount of a targeted contrast imaging agent, or a pharmaceutical composition thereof. The administration is preferably carried out under conditions that allow the contrast imaging agent to interact with an amyloid deposit present in the patient so that the interaction results in the binding of the contrast imaging agent to the amyloid deposit. After administration, the contrast imaging agent bound to amyloid deposits present in the patient is detected using an imaging technique, and one or more images of at least part of the body of the patient are generated.

In certain preferred embodiments, the inventive methods for detecting the presence of amyloid deposits in a system or in a patient are carried out by using a contrast imaging agent, wherein the imaging moiety comprises at least one metal-chelating moiety complexed to a paramagnetic metal ion; the detection is performed by Magnetic Resonance Imaging (MRI); and MR images are generated. Preferably, the paramagnetic metal ion is gadolinium III (Gd³⁺). In other preferred embodiments, the inventive methods are carried out by using a contrast imaging agent, wherein the imaging moiety comprises at least one metal-chelating moiety complexed to a radionuclide; the detection is performed by Single Photon Emission Computed Tomography (SPECT); and SPECT images are generated. Preferably, the radionuclide is technetium-99m (^{99m}Tc). In still other preferred embodiments, the inventive methods are carried out by using a contrast imaging agent, wherein the amyloid-binding moiety is labeled with a stable paramagnetic isotope; the detection is performed by Magnetic Resonance Spectroscopy (MRS); and MR images are generated. Preferably, the stable paramagnetic isotope is carbon-13 (¹³C) or fluorine-19 (¹⁹F).

In certain embodiments, the inventive methods are used to localize amyloid deposits in a patient. In other embodiments, the inventive methods are used to diagnose a pathophysiological condition associated with amyloid accumulation. In yet other embodiments, the methods are used to follow the progression of a pathophysiological condition associated with amyloid accumulation. In still other embodiments, the methods are used to monitor the response of a patient to a treatment for a pathophysiological condition associated with amyloid accumulation.

The bifunctional therapeutic molecules, targeted contrast imaging agents, pharmaceutical compositions, and methods described herein can also be used to diagnose, prevent or treat amyloid-related conditions affecting mammals other than humans. For example, they can be useful in the case of animal models for human amyloidoses as well as in animal Prion diseases, such as bovine spongiform encephalopathy in cattle; scrapie in sheep; transmissible encephalopathy in mink; and chronic wasting disease in muledeer and elk.

Other aspects, features and advantages of the present invention will become apparent from the following detailed description. It should be understood, however, that the detailed description and specific examples, while indicating preferred embodiments of the invention, are given by way of illustration only.

### BRIEF DESCRIPTION OF THE DRAWING

FIG. 1 shows the chemical structures of Congo red (FIG. 1A); Chrysamine-G (FIG. 1B); (trans,trans)-1-bromo-2,5-bis-(3-hydroxycarbonyl-4-hydroxy)-styrylbenzene (FIG. 1C); 4′-iodo-4′-deoxydoxorubicin (FIG. 1D); and Thioflavin T (FIG. 1E), which are known in the art to exhibit a high affinity for amyloid.

FIG. 2 shows the chemical structures of bathophenanthroline (FIG. 2A), bathocuproine (FIG. 2B), desferrioxamine (FIG. 2C), penicillamine (FIG. 2D), EDTA (FIG. 2E), EGTA (FIG. 2F), DTPA (FIG. 2G), TETA (FIG. 2H), TPEN (FIG. 2I), and α-lipoic acid (FIG. 2J), which are well-known metal chelators.

FIG. 3 shows the chemical structures of DOTA (FIG. 3A), TTHA (FIG. 3B), ECD (FIG. 3C), EDTMP (FIG. 3D), and HMPAO (FIG. 3E), which are known in the art to complex metal entity detectable by imaging techniques.

FIG. 4 shows the chemical structures of a family of new bifunctional molecules that comprise DTPA, acting as metal-chelating moiety, covalently linked to two identical amyloid-binding moieties, which are thioflavin derivatives. The parent molecule of the family (compound XH1) and different analogues are presented in FIG. 4A and FIG. 4B, respectively.

FIG. 5 shows results of the chemical characterization of compound XH1. The mass spectrum and ¹H-NMR spectrum of XH1 are presented in FIG. 5A and FIG. 5B, respectively.

FIG. 6 shows the chemical structures of a family of new bifunctional molecules that comprise α-lipoic acid, acting as metal-chelating moiety, covalently linked to one amyloid-binding moiety, a thioflavin derivative. The parent molecule of the family (compound XH2) and different analogues are presented in FIG. 6A and FIG. 6B, respectively.

FIG. 7 shows results of the chemical characterization of compound XH2. The mass spectrum and ¹H-NMR spectrum of XH2 are presented in FIG. 7A and FIG. 7B, respectively.

FIG. 8 is a graph showing the effects of the presence of the bifunctional molecule XH1, and of the metal-chelating compound DTPA, on the aggregation of Aβ₁₋₄₀. The aggregation is assessed by measure of the turbidity at 400 nm.

FIG. 9 shows the effects of XH1 on the viability of E17 rat cortical primary neurons (FIG. 9A) and on human SH-SY5Y neuroblastoma cells (FIG. 9B). The cell viability was assessed using the MTT assay and/or the LDH release assay 48 hours after treatment with XH1. The data are reported as mean cell-survival (% of untreated cultures)±standard deviation. At least three experiments were performed for each concentration of XH1.

FIG. 10 presents SDS-PAGE gels showing the effects of increasing concentration of XH1 on the expression of APP (FIG. 10A), and of different proteins used as controls: β-tubulin (FIG. 10A), APLP1 and APLP2 (FIG. 10B). Protein synthesis was measured 48 hours after SH-SY5Y human neuroblastoma cells were treated with XH1. A8717 was used as detecting antibody for APP.

FIG. 11 shows T1-weighted MRI signals measured from spherical phantoms incubated with a contrast imaging agent (Gd-XH1 or Gd-DTPA) in the presence or absence of Aβ₁₋₄₀ or HSA. In A1-5, Gd-XH1 is present at a concentration between 0 (A1) and 0.5 mM (A5). In B1-5, Gd-DTPA is present at a concentration between 0 (B1) and 1 mM (B5). All the spherical phantoms in lane C contain 0.025 mM of HSA and Gd-XH1, present at a concentration between 0 (C1) and 0.25 mM (C5). The spherical phantoms in lane D contain 0.5 mM of Gd-XH1 and between 0 (D1) and 0.025 mM (D5) of Aβ₁₋₄₀, while all the spherical phantoms in lane E contain 1 mM of Gd-XH1 and between 0 (E1) and 0.025 mM (E5) of Aβ₁₋₄₀. An increase in contrast imaging agent concentration led to a shorter T1, and therefore a brighter signal. No signal saturation was observed in these experiments.

FIG. 12 presents two graphs showing the variation of MRI signals from spherical phantoms as a function of contrast imaging agent (Gd-XH1 or Gd-DTPA) and protein (HSA or Aβ₁₋₄₀) present in the phantom. In FIG. 12A, the percent increase of R1 (i.e., 1/T1) is reported for both contrast imaging agents, Gd-XH1 and Gd-DTPA, as a function of concentration of Aβ₁₋₄₀. In FIG. 12B, R1 is reported for different concentrations of Gd-XH1 in the presence or the absence of HSA (0.025 mM).

FIG. 13 is a graph showing the variation of MRI signals reported as R1 (i.e., 1/T1) measured from spherical phantoms containing Gd-DTPA (0.25 mM) or Gd-XH1 (0.25 mM) and different concentrations of Aβ₁₋₄₂.

FIG. 14 shows MRI signals, which are enhanced in AD mouse and human brain tissue extracts when mixed with Gd-XH1 (0.025 mM).

FIG. 15 shows MRI images. The first series of images (presented in FIG. 15A) are baseline images of rat brains, showing anatomical features. The second series of images (presented in FIG. 15B) map the percent of increase in MRI signals measured about 1 hour after i.p. injection of Gd-XH1.

### DEFINITIONS

Throughout the specification, several terms are employed, that are defined in the following paragraphs.

The terms “amyloidosis” and “amyloid-related condition” are used herein interchangeably. They refer to any pathophysiological condition that affects humans or other mammals and is characterized by extracellular accumulation of amyloid in any organ or tissue of the body. Amyloidosis is associated with a wide range of medical disorders, but may also occur as a primary disease.

As used herein, the term “amyloid” refers to an aggregated (e.g., polymeric) form of an amyloid protein, the accumulation of which produces extracellular amyloid deposits. Regardless of the nature of the amyloid protein constituent, all amyloids share several properties: they form insoluble β-pleated sheet structures, that have a high affinity for Congo red, produce birefringence in polarized light, give a characteristic X-ray diffraction pattern, and are not susceptible to proteases.

As used herein, the term “amyloid deposit” refers to any insoluble quaternary structure formed by extracellular amyloid accumulation. Amyloid deposits can take the form of fibrils, filaments, plaques, and tangles.

The terms “amyloid protein” and “amyloidpeptide” are used herein interchangeably. They refer to an amyloid amino acid sequence in a monomeric (i.e., non-aggregated) form. Examples of amyloid (and amyloid-like) proteins include, but are not limited to, the amyloid immunoglobulin light chain (AL, associated with plasma cell dyscrasia, and found, for example, in patients with myelomatosis, i.e., bone marrow cancer); the serum amyloid associated protein (AA or SAP, associated with chronic inflammatory conditions, such as rheumatoid arthritis and osteomyelitis); the amyloid-β peptide (Aβ, which is associated with neurodegenerative disorders such as Alzheimer's disease, Down's syndrome, Lewy body dementia, hereditary cerebral hemorrhage with amyloidosis (Dutch type), and Guam Parkinson-Dementia; and may also accumulate in the brain of individuals with head injuries); the altered transthyretin (ATTR, associated with familial amyloidosis); the islet amyloid-polypeptide (IAPP or Amylin, which accumulates in the pancreas of patients with type II diabetes); and the prion protein (PrP, associated with the Prion diseases).

The terms “amyloid-β peptide”, “Aβ peptide”, and “Aβ” are used herein interchangeably. They are also known in the art as β-protein, β-A4 and A4. Aβ is a small, soluble, 4.3 kDa, 39-43 amino-acid long peptide, whose sequence has previously been published (see: C. Hilbich et al., J. Mol. Biol. 1992, 228: 460-473). In the present invention, the term amyloid-β peptide includes Aβ₁₋₄₃ as well as Aβ₁₋₄₂, Aβ₁₋₄₁, Aβ₁₋₄₀, and Aβ₁₋₃₉. The term “Aβ amyloid” refers to the amyloid-β peptide in an aggregated state. Deposits of Aβ amyloid are found, for example, in the brains of patients with Alzheimer's disease, adult patients with Down's syndrome, and occasionally individuals with head injuries.

The terms “binding affinity” and “affinity” are used herein interchangeably and refer to the level of attraction between molecular entities. Affinity can be expressed quantitatively as a dissociation constant (K_{d}), or its inverse, the association constant (Kₐ). In the context of this invention, two types of affinity are considered: (1) the affinity of an amyloid-binding moiety for amyloid deposits, and (2) the affinity of a metal-chelating moiety for a transition metal ion, or for another metal entity.

The term “amyloid-binding moiety” refers to any entity exhibiting high affinity and specificity for amyloid deposits. When an amyloid-binding moiety is part of a molecule, it confers its property to the molecule, and the molecule becomes “targeted” (i.e., it specifically and efficiently interacts with and binds to amyloid deposits). The binding between amyloid and an amyloid-binding moiety may be covalent or non-covalent (e.g., hydrophobic interactions, electrostatic interactions, dipole interactions, van der Waals interactions, hydrogen bonding, etc). Most often the binding is non-covalent.

The terms “metal-chelating” and “chelating”, as applied herein to chemical moieties, agents, compounds, or molecules refer to the ability of an entity characterized by the presence of two or more polar groups to participate in the formation of a complex (containing more than one coordinate bond) with a transition metal ion or another metal entity. Metal-chelating agents are known in the art. Examples of metal chelators, include, but are not limited to, bathocuproine, ethylenediaminetetraacetic acid, bathophenanthroline, desferrioxamine, and Clioquinol.

In the context of the present invention, the term “bifunctional molecule” refers to a molecule which comprises at least one metal-chelating moiety associated with at least one amyloid-binding moiety and which, consequently, exhibits a dual selectivity. More specifically, bifunctional molecules of the invention (1) bind with high affinity transition metal ions, and (2) display high affinity and specificity for amyloid deposits. The metal-chelating and amyloid-binding moieties may be associated by covalent or non-covalent bonds. Preferably, the association is covalent.

As used herein, the term “transition metal ion” refers to ionic forms of elements known in the art as transition metals. More particularly, in the context of the present invention, three biologically relevant transition metal ions are considered, namely: “zinc II”, “copper II”, and “iron III”, which, unless otherwise stated, refer to Zn²⁺, Cu²⁺, and Fe³⁺, respectively.

As used herein, the term “contrast imaging agent” refers to any entity that can be used to detect specific biological elements using imaging techniques. Contrast imaging agents of the invention are targeted molecules comprising at least one imaging moiety associated with at least one amyloid-binding moiety. In preferred embodiments of the invention, the imaging moiety in the contrast imaging agent comprises at least one metal-chelating agent complexed to a metal entity. Other contrast imaging agents of the invention comprise at least one metal-chelating moiety associated with at least one amyloid-binding moiety labeled with a stable paramagnetic isotope that is detectable by NMR. Contrast imaging agents of the invention can be used to detect amyloid deposits in in vitro, in vivo, and ex vivo systems as well as in living patients.

As used herein, the term “metal entity” refers to a paramagnetic metal ion that is detectable by imaging techniques such as Magnetic Resonance Imaging (MRI), or to a radionuclide, that is detectable by imaging techniques such as Single Photon Emission Computed Tomography (SPECT) and Positron Emission Tomography (PET).

A used herein, the term “paramagnetic metal ion” refers to a physiologically tolerable entity that can be complexed to a metal-chelating agent and is detectable by MRI. Preferably, the paramagnetic metal ion is selected from the group consisting of gadolinium III (Gd³⁺), chromium III (Cr³⁺), dysprosium III (Dy³⁺), iron III (Fe³⁺), manganese II (Mn²⁺), and ytterbium III (Yb³⁺).

As used herein, the term “radionuclide” refers to a radioactive isotope of a metallic element that can be complexed by a metal-chelating agent and used in radiopharmaceutical techniques. Preferred radionuclides are technetium-99m (^{99m}Tc), gallium-67 (⁶⁷Ga), yttrium-91 (⁹¹Y), indium-111 (¹¹¹In), rhenium-186 (¹⁸⁶Re), and thallium-201 (²⁰¹Tl).

As used herein, the term “stable paramagnetic isotope” refers to a paramagnetic nuclei that is detectable by nuclear magnetic resonance spectroscopy (MRS). Preferred stable paramagnetic isotopes for use in the present invention are carbon-13 (¹³C) and fluorine-19 (¹⁹F).

In the context of this invention, the term “redox active transition metal ion” refers to transition metal ions (such as Cu²⁺ and Fe³⁺), which can be reduced (to Cu⁺ and Fe²⁺, respectively), by engaging in a series of reactions that involve amyloid proteins and/or amyloid deposits and oxygen (O₂), and result in the formation of reactive oxygen species. Zinc II (Zn²⁺), which cannot undergo such reactions, is called a “redox inactive transition metal ion”.

As used herein, the term “reactive oxygen species” refers to molecules that derive from oxygen and generally are either toxic to biological systems or readily engage in reactions which produce toxic by-products. Reactive oxygen species include the superoxide radical anion (O₂.⁻), hydroxyl radical (.OH), hydrogen peroxide (H₂O₂), and singlet oxygen (¹O₂, ¹Δ_{g}).

The term “amyloid-mediated” when applied to the production of reactive oxygen species, refers to a series of processes which involve monomeric or polymeric forms of an amyloid protein, redox active transition metal ions, and oxygen, and result in the formation of reactive oxygen species.

“Oxidative stress” is a general term used herein to describe a system's state of damage that is directly or indirectly caused by amyloid-mediated production of reactive oxygen species. Oxidative stress occurs when the system's antioxidant defense mechanisms can no longer inhibit the deleterious action(s) of the reactive oxygen species produced. Oxidative stress which can first affect specific biomolecules (such as proteins, lipids and nucleic acids), ultimately induces massive cell damage that can result in cellular mutations, cell death, and tissue breakdown.

In the context of the present invention, the term “amyloid toxicity” refers to the ability of an amyloid protein to be toxic when aggregated in a β-sheet conformation, and/or to the ability of amyloid proteins and/or amyloid deposits to generate reactive oxygen species that have deleterious effects on a wide variety of biomolecules and can ultimately induce oxidative stress.

The term “prevention” is used herein to characterize a method that is aimed at delaying or preventing the onset of a pathophysiological condition associated with amyloid accumulation. The term “treatment” is used herein to characterize a method that is aimed at (1) delaying or preventing the onset of a condition associated with amyloidosis; or (2) slowing down or stopping the progression, aggravation, or deterioration of the symptoms of the condition; or (3) bringing about ameliorations of the symptoms of the condition; or (4) curing the condition. A treatment may be administered prior to the onset of the disease, for a prophylactic or preventive action. It may also be administered after initiation of the disease, for a therapeutic action.

The terms “individual” and “patient” are used herein interchangeably. They refer to a human or another mammal, that can be affected by a pathophysiological condition associated with amyloid accumulation but may or may not have such a disease.

As used herein, the term “system” refers to a biological entity that is known in the art to be able to produce and/or contain amyloid deposits. In the context of this invention, in vitro, in vivo, and ex vivo systems are considered; and the system may be a cell, a biological fluid, a biological tissue, or an animal. A system may, for example, originate from a live patient (e.g., it may be obtained by biopsy), or from a deceased patient (e.g., it may be obtained at autopsy). The patient may be a human or another mammal.

As used herein, the term “biological fluid” refers to a fluid produced by and obtained from a patient. Examples of biological fluids include, but are not limited to, cerebrospinal fluid (CSF), blood serum, urine, and plasma. In the present invention, biological fluids include whole or any fraction of such fluids derived by purification, for example, by ultrafiltration or chromatography.

As used herein, the term “biological tissue” refers to a tissue obtained from a patient. The biological tissue may be whole or part of any organ or system in the body (e.g., brain, pancreas, heart, kidney, gastrointestinal tract, thyroid gland, nervous system, skin, and the like).

As used herein, the term “effective amount” refers to any amount of a bifunctional molecule of the invention, or pharmaceutical composition thereof, that is sufficient to fulfill its intended purpose(s) (e.g., the purpose(s) may be: to delay or prevent the onset of a pathophysiological condition associated with amyloid accumulation; to slow down or stop the progression, aggravation, or deterioration of the symptoms of the condition; to bring about ameliorations of the symptoms of the condition; or to cure the condition. The purpose(s) may also be: to prevent, slow down, or stop amyloid accumulation in a system or a patient; to promote, induce, or otherwise facilitate dissolution of amyloid deposits present in the system or the patient; or to reduce, inhibit, or otherwise interfere with amyloid-mediated production of reactive oxygen species).

As used herein, the term “imaging effective amount” refers to any amount of a contrast imaging agent of the invention, or pharmaceutical composition thereof, that is sufficient to allow the detection, using an imaging technique, of amyloid deposits present in a system or in a patient.

A “pharmaceutical composition”, as used herein, is defined as comprising at least one reagent of the invention (bifunctional therapeutic molecule or targeted contrast imaging agent), or a physiologically tolerable salt thereof, and at least one pharmaceutically acceptable carrier.

The term “physiologically tolerable salt” refers to any acid addition or base addition salt that retains the biological activity and properties of the free base or free acid, respectively, and that is not biologically or otherwise undesirable. Acid addition salts are formed with inorganic acids (e.g., hydrochloric, hydrobromic, sulfuric, nitric, phosphoric acids, and the like); and organic acids (e.g., acetic, propionic, pyruvic, maleic, malonic, succinic, fumaric, tartaric, citric, benzoic, mandelic, methanesulfonic, ethanesulfonic, p-toluenesulfonic, salicylic acids, and the like). Base addition salts can be formed with inorganic bases (e.g., sodium, potassium, lithium, ammonium, calcium, magnesium, zinc, aluminum salts, and the like) and organic bases (e.g., salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethyl-aminoethanol, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like).

As used herein, the term “pharmaceutically acceptable carrier” refers to a carrier medium which does not interfere with the effectiveness of the biological activity of the active ingredients and which is not excessively toxic to the hosts at the concentrations at which it is administered. The term includes solvents, dispersion media, coatings, antibacterial and antifungal agents, isotonic agents, absorption delaying agents, and the like. The use of such media and agents for pharmaceutically active substances is well known in the art (see, for example, Remington's Pharmaceutical Sciences, E. W. Martin, 18^{th} Ed., 1990, Mack Publishing Co., Easton, Pa.).

The terms “(C₁-C₆)alkyl” and “(C₁-C₁₂)alkyl” as used herein, refer to saturated, straight- or branched-chain hydrocarbon radicals containing between one and six and one and twelve carbon atoms, respectively. Examples of C₁-C₆ alkyl radicals include, but are not limited to, methyl, ethyl, propyl, isopropyl, n-butyl, tert-butyl, neopentyl and n-hexyl radicals. Examples of C₁-C₁₂ alkyl radicals includee, but are not limited to, methyl, ethyl, n-propyl, isopropyl, n-hexyl, n-octyl, n-decyl and n-dodecyl radicals.

The terms “halo” and “halogen,” as used herein, refer to an atom selected from fluorine, chlorine, bromine and iodine.

The term “haloalkyl” denotes an alkyl group, as defined above, having one, two or three halogen atoms attached thereto and is exemplified by such groups as chloromethyl, bromoethyl, trifluoromethyl, and the like.

The term “aryl”, as used herein, refers to a mono-, bicyclic or tricyclic carbocyclic ring system having one, two or three aromatic rings including, but not limited to, phenyl, naphthyl, anthryl, azulyl, tetrahydronaphthyl, indanyl, indenyl and the like.

The term “substituted aryl,” as used herein, refers to an aryl group, as defined herein, substituted by independent replacement of one or more of the hydrogen atoms thereon with substituents independently selected from substituents such as, but not limited to, alkyl, haloalkyl, alkoxy, thioalkoxy, amino, alkylamino, dialkylamino, acylamino, hydroxy, cyano, halo, mercapto, nitro, carboxaldehyde, carboxyl, alkoxycarbonyl, carboxamide, aryl, substituted aryl, heteroaryl, substituted heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl. Representative substituents include, but are not limited to, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CH₂OH, CH₂CH₂OH, CH₂NH₂, CH₂SO₂CH₃, tetrafluorophenyl and pentafluorophenyl.

The term “heteroaryl,” as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

The term “substituted heteroaryl,” as used herein, refers to a heteroaryl group as defined herein, substituted by independent replacement of one, or more of the hydrogen atoms thereon with substituents independently selected from substituents such as, but not limited to, alkyl, haloalkyl, alkoxy, thioalkoxy, amino, alkylamino, dialkylamino, acylamino, hydroxy, cyano, halo, mercapto, nitro, carboxaldehyde, carboxyl, alkoxycarbonyl, carboxamide, aryl, substituted aryl, heteroaryl, substituted heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl. Representative substituents include, but are not limited to, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CH₂OH, CH₂CH₂OH, CH₂NH₂, CH₂SO₂CH₃, tetrafluorophenyl and pentafluorophenyl.

The term “C₃-C₁₂-cycloalkyl”, as used herein, denotes a monovalent group derived from a monocyclic or bicyclic saturated carbocyclic ring compound having from 3 to 12 carbon atoms by the removal of a single hydrogen atom. Examples include, but not limited to, cyclopropyl, cyclobutyl, cyclopentyl, cyclohexyl, bicyclo [2.2.1]heptyl, and bicyclo [2.2.2] octyl.

The term “substituted C₃-C₁₂-cycloalkyl”, as used herein, refers to a C₃-C₁₂-cycloalkyl group, as defined herein, substituted by independent replacement of one or more of the hydrogen atoms therein with substituents independently selected from substituents such as, but not limited to, alkyl, haloalkyl, alkoxy, thioalkoxy, amino, alkylamino, dialkylamino, acylamino, hydroxy, cyano, halo, mercapto, nitro, carboxaldehyde, carboxyl, alkoxycarbonyl, carboxamide, aryl, substituted aryl, heteroaryl, substituted heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl. Representative substituents include, but are not limited to, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CH₂OH, CH₂CH₂OH, CH₂NH₂, CH₂SO₂CH₃, tetrafluorophenyl and pentafluorophenyl

The term “heterocycloalkyl,” as used herein, refers to a non-aromatic 3-, 4-, 5-, 6- or 7-membered ring or a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “substituted heterocycloalkyl”, as used herein, refers to a cycloalkyl group, as gefined herein, substituted by independent replacement of one or more of the hydrogen atoms therein with substituents independently selected from substituents such as, but not limited to, alkyl, haloalkyl, alkoxy, thioalkoxy, amino, alkylamino, dialkylamino, acylamino, hydroxy, cyano, halo, mercapto, nitro, carboxaldehyde, carboxyl, alkoxycarbonyl, carboxamide, aryl, substituted aryl, heteroaryl, substituted heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl. Representative substituents include, but are not limited to, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CH₂OH, CH₂CH₂OH, CH₂NH₂, CH₂SO₂CH₃, tetrafluorophenyl and pentafluorophenyl.

The term “heterocyclic”, as used herein, refers to heterocycloalkyl and heteroaryl. The term “substituted heterocyclic”, as used herein, refers to substituted heterocycloalkyl and substituted heteroaryl.

The term “C₁-C₆ alkoxy”, as used herein, refers to a C₁-C₆ alkyl group, as previously defined, attached to the parent molecular moiety through an oxygen atom. Examples of C₁-C₆-alkoxy include, but are not limited to, methoxy, ethoxy, propoxy, isopropoxy, n-butoxy, tert-butoxy, neopentoxy and n-hexoxy.

The term “C₁-C₃-alkylamino”, as used herein, refers to one or two C₁-C₃-alkyl groups, as previously defined, attached to the parent molecular moiety through a nitrogen atom. Examples of C₁-C₃-alkylamino include, but are not limited to, methylamino, dimethylamino, ethylamino, diethylamino, and propylamino.

The term “alkylamino” refers to a group having the structure —NH(C₁-C₁₂ alkyl) where C₁-C₁₂ alkyl is as previously defined.

The term “dialkylamino” refers to a group having the structure —N(C₁-C₁₂ alkyl) (C₁-C₁₂ alkyl), where C₁-C₁₂ alkyl is as previously defined. Examples of dialkylamino are, but not limited to, dimethylamino, diethylamino, methylethylamino, piperidino, and the like.

The term “alkoxycarbonyl” represents an ester group, i.e. an alkoxy group, attached to the parent molecular moiety through a carbonyl group such as methoxycarbonyl, ethoxycarbonyl, and the like.

The term “carboxy”, as used herein, refers to a group of formula —COOH.

The term “carboxamido”, as used herein, refers to a group of formula —C(O)NH(C₁-C₁₂ alkyl) or —C(O)N(C₁-C₁₂ alkyl) (C₁-C₁₂ alkyl).

The term “cycloalkylamino” as used herein refers to a group having the structure N(C₃₋₁₂ cycloalkyl) where C₃₋₁₂cycloalkyl is as previously defined.

The term “aryloxy” as used herein refers to a group having the structure [—O-aryl], where aryl is as previously defined.

The term “substituted aryloxy” as used herein refers to a group having the structure [—O-substituted aryl], where substituted aryl is as previously defined.

The term “heteroaryloxy” as used herein refers to a group having the structure [—O-heteroaryl], where heteroaryl is as previously defined.

The term “substituted heteroaryloxy” as used herein refers to a group having the structure [—O— substituted heteroaryl], where substituted heteroaryl is as previously defined.

The term “heterocycloalkyloxy” as used herein refers to a group having the structure [—O-heterocycloalkyl], where heterocycloalkyl is as previously defined.

The term “substituted heterocycloalkyloxy” as used herein refers to a group having the structure [—O-substituted heterocyloaklyl], where substituted heterocycloalkyl is as previously defined.

The term “thioalkyl” as used herein refers to a group having the structure [—S-alkyl], where alkyl is as previously defined.

The term “sulfonamido” refers to a group having the structure —S(O)(O)NH₂, —S(O)(O)NH(C₁-C₁₂alkyl) or —S(O)(O)N(C₁-C₁₂alkyl)(C₁-C₁₂alkyl), where (C₁-C₁₂alkyl) is as previously defined.

Additional definitions are provided throughout the Detailed Description.

### DETAILED DESCRIPTION OF CERTAIN PREFERRED EMBODIMENTS

The present invention is directed to the diagnosis, prevention, and treatment of pathophysiological conditions associated with amyloid accumulation. In particular, the invention encompasses reagents and strategies for detecting the presence of amyloid deposits, and for preventing or treating amyloid-related conditions. In certain preferred embodiments, the invention allows the diagnosis, prevention, and treatment of pathophysiological conditions associated with aggregation and accumulation of amyloid and amyloid-like proteins in the brain.

I. Bifunctional Therapeutic Molecules

One aspect of the present invention relates to a new class of targeted therapeutic reagents.

Investigation of the roles of biometals in the brains of normally aging individuals and of Alzheimer's patients have helped set a new direction for the development of more effective treatments. Regulation of the levels of transition metal ions using metal-chelating molecules has been shown to dissolve amyloid deposits and prevent oxidative damage in vitro. Metal chelators have also been found to significantly reduce amyloid load in the brains of transgenic mice (R. A. Chemy et al., Neuron. 2001, 30: 665-676). These results are very promising and demonstrate the potential of such an approach for the treatment of amyloid-related conditions. However, most known metal chelators are non-specific and can interfere with the normal physiological function(s) of other metal-requiring biomolecules, thereby creating side effects that may prove too great for clinical use.

The present invention encompasses the recognition that targeted metal-chelating agents capable of preventing metal ions from interacting with amyloid proteins and amyloid deposits without perturbing the action of other important biomolecules, should exhibit less undesirable side-effects and be more effective than most of the non-specific metal chelators that are currently used, tested, or proposed as therapeutics for the treatment of amyloid-related pathophysiological conditions. Accordingly, the invention provides therapeutic reagents designed to (1) have some degree of attraction for amyloid, and (2) act as metal chelators. More specifically, the present invention provides bifunctional molecules comprising at least one metal-chelating moiety associated with at least one amyloid-binding moiety.

Amyloid-Binding Moieties

Amyloid-binding moieties are entities that have some degree of attraction for amyloid deposits and can play a targeting role when comprised in a bifunctional molecule. In preferred embodiments, amyloid-binding moieties exhibit high affinity and specificity for amyloid deposits, i.e., they specifically and/or efficiently interact with, bind to, or label amyloid. Preferably, amyloid-binding moieties are stable, non-toxic entities that retain their binding properties under in vitro and in vivo conditions. In certain preferred embodiments, amyloid-binding moieties have a high affinity and specificity for Aβ amyloid. More specifically, these amyloid-binding entities bind Aβ with a dissociation constant (K_{d}) between 0.1 nM and 10 μM when determined using synthetic Aβ peptides or Alzheimer's disease brain tissue (as described in the Examples section). In other preferred embodiments, amyloid-binding moieties are capable of crossing the blood-brain barrier. This property is particularly important when the bifunctional molecule is to be used as therapeutic agent for the treatment of neurodegenerative disorders characterized by accumulation of aggregated amyloid and amyloid-like proteins in the brain.

The interaction between an amyloid-binding moiety and amyloid deposits may be covalent or non-covalent. Most often, the interaction between an amyloid-binding moiety and amyloid deposits is non-covalent (see below). Examples of non-covalent interactions include, but are not limited to, hydrophobic interactions, electrostatic interactions, dipole interactions, van der Waals interactions, and hydrogen bonding. Irrespective of the nature of the interaction, the binding between amyloid deposits and an amyloid-binding moiety within a bifunctional molecule of the invention should be selective, specific, and strong enough to allow the metal-chelating moiety to play its role (i.e., to prevent, inhibit, or reverse interactions between transition metal ions and amyloid proteins and/or amyloid deposits).

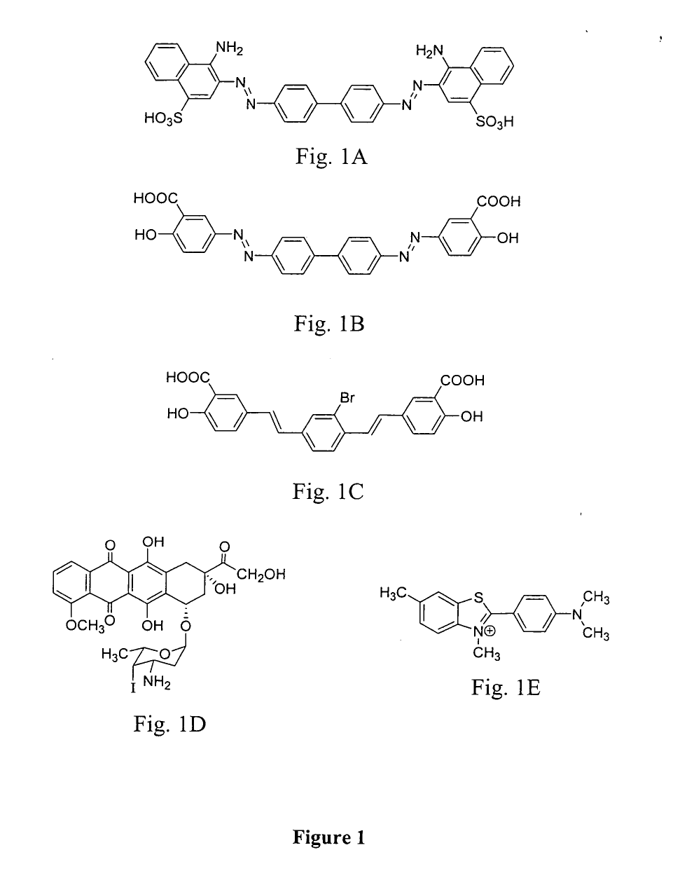

Suitable amyloid-binding moieties for use in the present invention include any of the amyloid-binding entities that fulfill the requirements listed above. Actually, the development of biological markers of amyloid deposits has been a research goal for several years (W. E. Klunk, Neurobiol. Aging, 1998, 19: 145-147) and a large number of such compounds are now available. Congo red (whose chemical structure is presented in FIG. 1A) has been used for several decades to stain amyloid deposits in vitro (M. Tubis et al., J. Am. Pharm. Assoc. 1960, 49: 422-425; M. Tubis et al., Nukl. Med. 1962, 3: 25-38).

Several models have been proposed to explain the specific affinity of Congo red for amyloid proteins aggregated in a β-sheet conformation (W. E. Klung et al., J. Histochem. Cytochem. 1989, 37: 1273-1281; W. E. Klunk et al., Neurol. Aging, 1994, 15: 691-698; D. B. Carter and K.-C. Chou, Neurol. Aging, 1998, 19: 37-40). In all the proposed models, a stoichiometric and saturable electrostatic interaction between the anionic sulphonate groups of Congo red and basic amino acids such as arginine and lysine on the amyloid peptide is believed to play an important role in the preferential binding. In some of the proposed models, an aromatic interaction between the biphenyl moiety of Congo red and the phenylalanine residues of the amyloid peptide is also hypothesized to be involved in the interaction.

Based on these models, it was concluded that molecules that bind specifically to amyloid deposits tend to be long, conjugated systems possessing multiple phenyl rings with negatively charged groups at each end. Using these criteria, bisdiazobenzidine compounds related to Congo red (see, for example, U.S. Pat. Nos. 4,933,156; 5,008,099; and 5,039,511); Chrysamine-G (presented in FIG. 1B) and derivatives (see, for example, U.S. Pat. Nos. 6,114,175; 6,133,259; and 6,168,776); (trans, trans)-1-bromo-2,5-bis-(3-hydroxycarbonyl-4-hydroxy)-styrylbenzene (BSB) (FIG. 1C) and a variety of analogues have been designed, synthesized (N. A. Dezutter et al., Eur. J. Nucl. Med. 1999, 26: 1392-1399; D. M. Skovronsky et al., Proc. Natl. Acad. Sci. U.S.A., 2000, 97: 7609-7614), radiolabeled and evaluated as potential in vitro and in vivo probes of amyloid deposition (W. E. Klunk et al., Neurobiol. Aging, 1994, 15: 691-698; W. Zhen et al., J. Med. Chem. 1999, 42: 2805-2815; N. A. Dezutter et al., J. Nucl. Med. 1999, 26: 1392-1399; D. M. Skovronsky et al., Proc. Natl. Acad. Sci. USA, 2000, 97: 7609-7614). All these molecules were found to possess a high affinity and specificity for Aβ and are therefore suitable for use in the present invention. However the presence of highly polar functional groups on some of these biological markers was demonstrated to limit their entry into the brain and only the derivatives that do not have polar functional groups were shown to display high brain uptake. The design of an inventive bifunctional molecule will be dictated by its intended purpose(s) and amyloid-binding moieties will be chosen based on their known, observed or expected properties (for example, their blood-brain barrier permeability).

When selecting an amyloid-binding moiety to design a bifunctional therapeutic molecule, toxicity should also be a factor to consider. For example, it is known in the art that azo dyes may be carcinogenic (D. L. Morgan et al., Environ. Health Perspec. 1994, 102: 63-78). The potential carcinogenicity of azo dyes is believed to result from their extensive metabolic degradation to the free (toxic) parent amine by intestinal bacteria (C. E. Cerniglia et al., Biochem. Biophys. Res. Comm. 1982, 107: 1224-1229; C. E. Cemiglia et al., Carcinogen. 1982, 3: 1255-1260). To avoid toxicity problems, it may be desirable to avoid azo dyes such as Congo red, Chrysamine-G and their derivatives as amyloid-binding moieties, or to select a route of administration such that the bifunctional therapeutic molecule by-passes intestinal bacteria.

Smaller molecules have also been evaluated for their ability to specifically bind to amyloid deposits. These include, but are not limited to, the anthracycline 4′-iodo-4′-deoxydoxorubicin (depicted in FIG. 1D), which has been found to strongly bind to different types of amyloid proteins and amyloid deposits (G. Merlini et al., Proc. Natl. Acad. Sci. USA, 1995, 92: 2959-2963), and thiazole dyes such as Primulin, Thioflavin S, and Thioflavin T (whose chemical structure is shown in FIG. 1E). These thiazole dyes are known to stain amyloid in tissue sections and to efficiently bind synthetic Aβ in vitro (G. Kelenyi, Histochem. Cytochem. 1967, 15: 172-180; J. Burns et al., J. Pathol. Bacteriol. 1967, 94: 337-344; R. Guntem et al., Experientia 1992, 48: 8-10; H. LeVine, Meth. Enzymol. 1999, 309: 274-284). Interestingly, removal of the methyl group from the heterocycle of Thioflavin T, which eliminates the positive charge, has recently been shown to provide a series of lipophilic dyes with high affinity for Aβ as well as good brain uptake in rodents (W. E. Klunk et al., Life Sci. 2001, 69: 1471-1484; Z. P. Zhuang et al., J. Med. Chem. 2001, 44: 1905-1914).

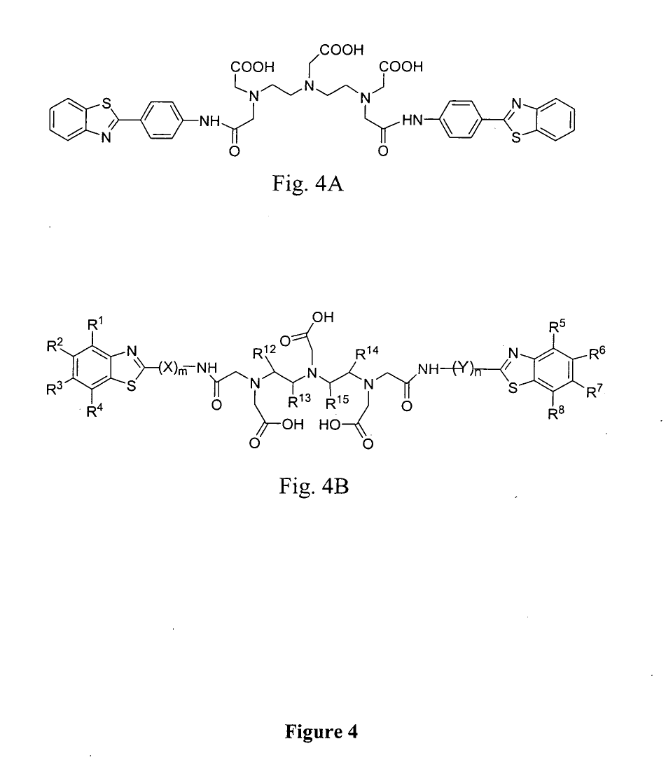

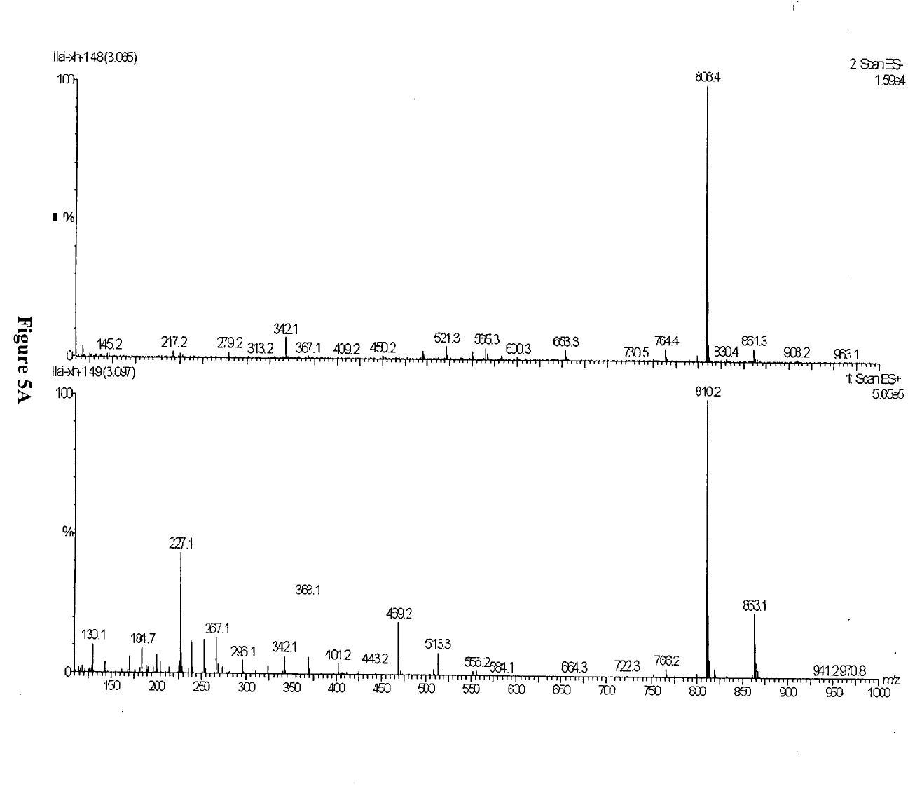

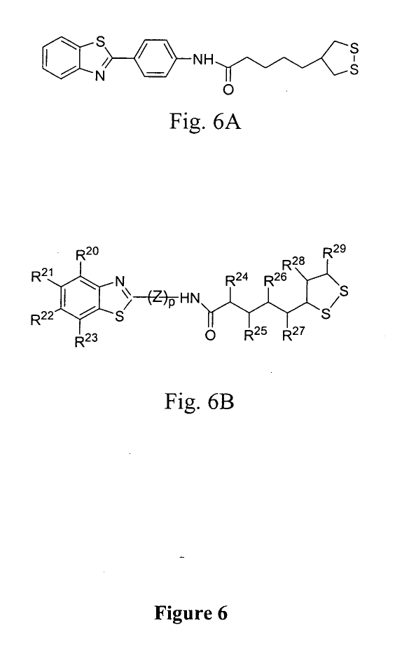

In certain preferred embodiments, amyloid-binding moieties are derivatives of small molecules that have been reported to exhibit high affinity for amyloid deposits and are capable of crossing the blood-brain barrier. (D. M. Skovronsky et al., Proc. Natl. Acad. Sci. U.S.A., 2000, 97: 7609-7614). Example 1 and Example 9 describe the synthesis of two families of novel bifunctional molecules that comprise at least one of such an amyloid-binding small molecule. The parent compounds of these families are presented on FIG. 4(A) and FIG. 6(A), respectively.

It should be apparent that the modification of the diethylene triamine pentaacetic acid (DTPA) backbone of the molecule can affect the solubility, the permeability, and the metal binding properties of the molecule in the claimed applications. Examples of this include dicyclohexyl-diethylenetriaminepentaacetic acid (Cy₂DTPA) (see, for example, T. J. Caulfield, et al., Bioorg. Med. Chem. Lett., 1995, 5: 1657-1662), wherein the polyamine chain is substituted by having cyclohexyl rings incorporated into the backbone; 4-carboxy-5,8,11-tris(carboxymethyl)-1-phenyl-2-oxa-5,8,11-triazatridecan-13-oic acid; (BOPTA; F. Uggeri, et al., Inorg. Chem., 1995, 34: 633-642), COPTA, 4-ethoxybenzyl diethylenetriamine pentaacetic acid (EOB-DTPA; G. Schumann-Giampieri et al., Pharmacol. Sciences, 1993, 799-806), and B-21327-7, wherein the polyamine chain is partially substituted; and others. For reviews of the synthesis and properties of such structures see, for example, S. Laurent, et al., Helv. Chim. Acta, 2000, 83: 394-406 and, P. Caravan, et al., Chem. Rev., 1999, 99: 2293-2352, R. Lauffer, Chem. Rev., 1987, 87: 901-927), and references contained therein.

Preferably, the amyloid-binding moieties contain at least one functional group that can be used (or is easily chemically converted to a different functional group that can be used) to covalently attach the amyloid-binding moieties to metal-chelating moieties. Suitable functional groups include, but are not limited to, amines (preferably primary amines), thiols, carboxy groups, and the like.

Metal-Chelating Moieties

Metal-chelating moieties are entities that can bind with high affinity transition metal ions. Preferably, the transition metal ions that can be complexed by the metal-chelating moieties are biometals (i.e., they are biologically relevant transition metal ions). Most preferably, the metal-chelating moieties bind with high affinity transition metal ions that are found highly concentrated in and around amyloid deposits. In certain preferred embodiments, metal-chelating moieties bind with high affinity at least one transition metal ion selected from the group consisting of zinc II (Zn²⁺), copper II (Cu²⁺), and iron III (Fe³⁺). Preferably, metal-chelating moieties are stable, non-toxic entities that retain their binding property under in vitro and in vivo conditions.

Experimental evidence accumulates that implicates transition metal ions in at least two aspects of the pathology of amyloid-related clinical conditions. Studies indicate (1) that interactions between transition metals and amyloid proteins may potentiate amyloid toxicity by accelerating the aggregation and accumulation of amyloid peptides into the toxic β-sheet conformation and (2) that redox active transition metal ions may increase the amyloid toxicity by favoring the production of reactive oxygen species.

In particular, the amyloid-β peptide has been shown to possess selective high and low affinity Cu²⁺ and Zn²⁺ binding sites (A. I. Bush et al., J. Biol. Chem. 1994, 269: 12152-12158) and the interaction of Aβ with Cu²⁺, Zn²⁺ and Fe³⁺ has been demonstrated to promote the aggregation and accumulation of the peptide (A. I. Bush et al., J. Biol. Chem. 1994, 265: 1464-1467). The recognition, in the Applicant's laboratory, that metal chelators reverse the aggregation of synthetic Aβ peptides, dissolve amyloid in post-mortem human brain specimens (C. S Atwood et al., J. Biol. Chem. 1998, 273: 12817-12826; X. Huang et al., J. Biol. Chem. 1997, 272: 26464-26470; R. A. Cherny et al., J. Biol. chem. 1999, 274: 23223-23228) and induce a marked inhibition of amyloid load in the brain of Tg2576 transgenic mouse model for Alzheimer's disease (R. A. Cherny et al., Neuron. 2001, 30: 665-676) has led to proposals of metal chelators and metal complexing molecules as potential therapeutic agents for the treatment of pathophysiological conditions associated with amyloid accumulation (see U.S. Pat. No. 6,323,218).

By interfering with the interaction(s) between transition metal ions and amyloid proteins and/or amyloid deposits, metal chelators can have an effect on amyloid toxicity. According to this aspect of the invention, suitable metal-chelating moieties are entities that can reduce or inhibit amyloid toxicity by preventing, slowing down or stopping the aggregation and accumulation of amyloid proteins, and/or by promoting, inducing, or otherwise facilitating dissolution of amyloid deposits. This can, for example, be achieved when the metal-chelating moiety binds with high affinity at least one transition metal ion selected from the group consisting of zinc II (Zn²⁺), copper II (Cu²⁺), and iron III (Fe³⁺).

There is mounting evidence that oxidative stress causing cellular damage is critical to the neurodegeneration observed in Alzeimer's disease (R. N. Martins et al., J. Neurochem. 1986, 46: 1042-1045). An increase in oxidation of proteins as well as nuclear and mitochondrial DNA is consistently observed in the brains of AD patients (P. Gabbita et al., J. Neurochem. 1998, 71: 2034-2040; W. R. Markesbery, Free Radic. Biol. Med. 1997, 23: 134-147; M. P. Cuajungco et al., J. Biol. Chem. 2000, 275: 19439-19442). Furthermore, the amyloid-β peptide has been demonstrated to have the ability to enhance the generation of reactive oxygen species in cells of neural origin as well as in cell-free media (C. Behl et al., Cell, 1994, 77: 817-827; X. Huang et al., J. Biol. Chem. 1999, 274: 37111-37116; X. Huang et al., Biochem. 1999, 38: 7609-7616). Extensive redox chemical reactions were observed to take place when Aβ binds Cu²⁺ and/or Fe³⁺, reducing the oxidation state of both metals and producing H₂O₂ from O₂ in a catalytic manner (X. Huang et al., Biochem. 1999, 38: 7609-7616). Because elevated levels of copper (400 μM), zinc (1 mM) and iron (1 mM) are found in amyloid deposits in AD-affected brains (M. A. Lovell et al., J. Neurol. Sci. 1998, 158: 47-52; M. A. Smith et al., Proc. Natl. Acad. Sci. USA 1997, 94: 9866-9868), the oxidative stress observed in Alzheimer's disease is believed to be related to the production of reactive oxygen species by metal-bound forms of Aβ. This hypothesis is supported by the recent observation that senile plaques and neurofibrillary tangles isolated from AD brains were capable of generating reactive oxygen species, and that the presence of copper and iron was found to be necessary for the reaction to take place (L. M. Sayre et al., J. Neurochem. 2000, 74: 270-279).

Redox active metals, such as Cu²⁺ and Fe³⁺, can engage in reactions which result in the production of reactive oxygen species (W. R. Markesbery, Free Rad. Biol. Med. 1997, 23: 134-147). One series of such reactions is shown below. The amyloid-β peptide as well as Aβ amyloid have the ability of reducing Cu²⁺ (or Fe³⁺) and simultaneously form hydrogen peroxide (H₂O₂) from the apparent reduction of molecular oxygen (O₂) to the superoxide radical anion (O₂.⁻). This process is followed by a Fenton-like reaction, which generates hydroxyl radicals.
reduced compound+O₂→oxidized compound+O₂.⁻ (a)
e.g.: Fe²⁺+O₂→Fe³⁺+O₂.⁻, or
Cu⁺+O₂→Cu²⁺+O₂.⁻
   2O₂.⁻+2H⁺→H₂O₂+O₂ (b)
   reduced compound+H₂O₂→oxidized compound+.OH+OH⁻ (c)
e.g.: Fe²⁺+H₂O₂→Fe³⁺+.OH+OH⁻, or
Cu⁺+H₂O₂→Cu^{2++.OH+OH}⁻

In addition to the reactive oxygen species mentioned above, other free radicals can be formed and also contribute to the pathology of amyloidoses. These include, but are not limited to, radical forms of the amyloid peptides and amyloid deposits, and peroxynitrite, which can be, for example, produced by the reaction of the superoxide radical anion with nitric oxide.

According to this aspect of the present invention, suitable metal-chelating moieties are entities that can reduce or inhibit amyloid toxicity by reducing, inhibiting, or otherwise interfering with the biometal- and amyloid-mediated production of reactive oxygen species, including the superoxide radical anion (O₂.⁻), hydrogen peroxide (H₂O₂), hydroxyl radical (.OH) and singlet oxygen (¹O₂). This can be achieved when the metal-chelating moiety in the bifunctional molecule binds with high affinity at least one redox active transition metal ion selected from the group consisting of copper II (Cu²⁺) and iron III (Fe³⁺).

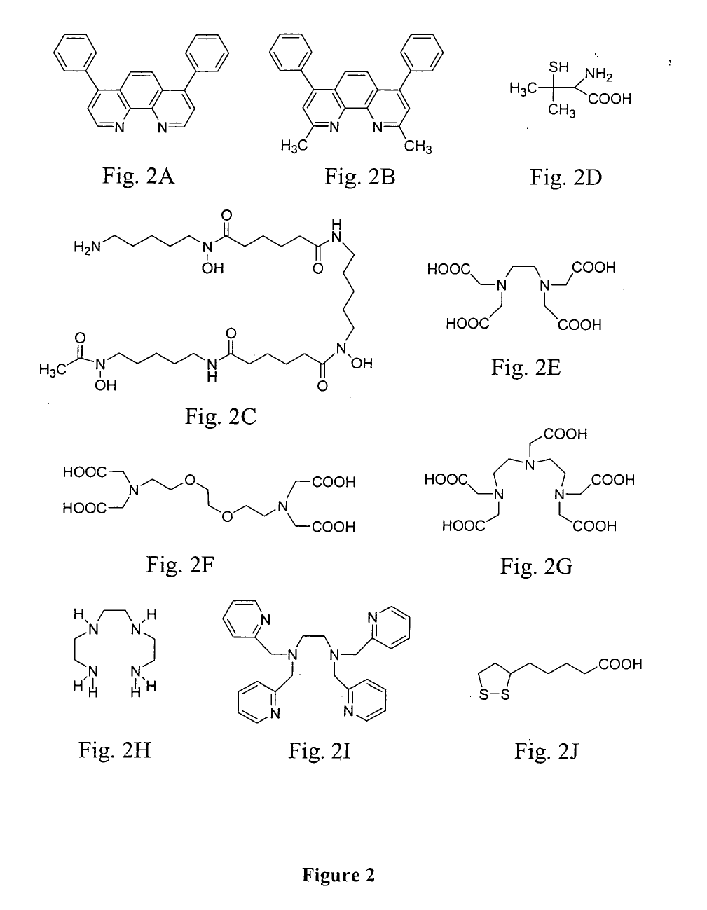

Suitable metal-chelating moieties for use in the present invention may be any of a large number of metal chelators and metal complexing molecules known to bind with high affinity transition metal ions. Those include, but are not limited to, aromatic amines such as bathophenanthroline (4,7-diphenyl-1,10-phenanthroline, whose chemical structure is presented in FIG. 2A); bathocuproine (2,9-dimethyl-4,7-diphenyl-1,10-phenanthroline, FIG. 2B), and TPEN (tetrakis-(2-pyridylmethyl) ethylenediamine, FIG. 21); and aliphatic amines such as deferrioxamine (FIG. 2C), penicillamine (2-amino-3-mercapto-3-methylbutanoic acid, FIG. 2D), EDTA (ethylenediamine-tetraacetic acid, FIG. 2E), EGTA (O,O′-bis(2-aminoethyl)-ethyleneglycol-N,N′,N″,N′″-tetraacetic acid, FIG. 2F), DTPA (diethylene triaminepentaacetic acid, FIG. 2G), and TETA (triethylene tetramine, FIG. 2H); and functional derivatives, homologues and analogues thereof.

Alpha-lipoic acid derivatives constitute another family of metal-chelating agents that can be used in the practice of the present invention. In addition to exhibiting metal-chelating properties, α-lipoic acid derivatives also have a powerful anti-oxidant activity (for a review, see, for example, H. Moini et al., Toxicol. Appl. Pharmacol. 2002, 182: 84-90; or G. Biewenga et al., Gen. Pharmac. 1997, 29: 315-331). The anti-oxidative effects of α-lipoic acid have been demonstrated both in neuronal and non-neuronal tissues (M. A. Lynch, Nutr. Neurosci. 2001, 4: 419-438). Results from in vitro, animal, and preliminary human studies indicate that lipoic acids may be effective in numerous neurodegenerative disorders (L. Packer et al., Free Radic. Biol. Med. 1997, 22: 359-378). In particular, α-lipoid acids have been shown to be efficient at reducing neuronal amyloid burden in post-mortem human brains of AD patients (J. Fonte et al., J. Alzheimer Dis. 2001, 3: 209-219) and to reverse memory impairment and brain oxidative stress in aged mice (S. A. Farr et al., J. Neurochem. 2003, 83: 1173-1183).

The additional (i.e., anti-oxidant) property exhibited by α-lipoic acid derivatives (compared to other metal chelating agents) is expected to broaden the range of action of the bifunctional molecule, as α-lipoic acids can exert their antioxidative effects through different mechanisms including by chelating metal ions, by scavenging reactive oxygen species (ROS) or other radicals, by regenerating endogenous antioxidants (such as vitamin C, vitamin E and glutathione), and/or by repairing oxidative damage.

The precise design of an inventive bifunctional molecule will be influenced by its intended purpose(s) and metal-chelating moieties will be selected based on their known, observed or expected properties. Preferred metal-chelating moieties for interfering with aggregation of amyloid proteins and for promoting dissolution of amyloid deposits include DTPA, bathocuproine, bathophenanthroline, penicillamine, and derivatives, homologues, and analogues thereof, or any combinations thereof. Preferred metal-chelating moieties for interfering with the biometal- and amyloid-mediated production of reactive oxygen species include bathocuproine, bathophenanthroline, α-lipoic acid and derivatives, homologues and analogues thereof, or any combinations thereof.

A first family of new bifunctional molecules comprising DTPA as metal chelating moiety, covalently linked to two identical amyloid-binding moieties (a benzothiazole derivative), has been developed and their synthesis, properties and uses are described in the Examples section (see Examples 1 and 4 to 6). The synthesis of a second family of bifunctional molecules comprising α-lipoic acid, acting as metal-chelating moiety, covalently linked to one amyloid-binding moiety, (selected from the same benzothiazole derivatives that those used in the first family), is described in Example 9.

Preferably, the metal-chelating moieties contain at least one functional group that can be used (or can be easily chemically converted to a different functional group that can be used) to covalently attach the metal-chelating moieties to amyloid-binding moieties. Suitable functional groups include, but are not limited to, amines (preferably primary amines), thiols, carboxy groups, and the like.

Synthesis of Bifunctional Molecules

The inventive bifunctional molecules may be prepared by any synthetic method known in the art, the only requirement being that, after reaction, the amyloid-binding and metal-chelating moieties retain their binding and chelating properties, respectively. The amyloid-binding moieties may be associated with the metal-chelating moieties in a variety of ways. Preferably, the amyloid-binding moieties are covalently attached to the metal-chelating moieties. As can be appreciated by those skilled in the art, the amyloid-binding and metal-chelating moieties may be attached to each other either directly or through a linker.

In certain preferred embodiments, the metal-chelating and amyloid-binding moieties are directly covalently linked to each other. The direct covalent binding can be through an amide, ester, carbon-carbon, disulfide, carbamate, ether, thioether, urea, amine, or carbonate linkage. The covalent binding can be achieved by taking advantage of functional groups present on the amyloid-binding and metal-chelating moieties. Suitable functional groups that can be used to attach the two moieties together include, but are not limited to, amines (preferably primary amines), anhydrides, hydroxy groups, carboxy groups, and thiols. For example, as described in Example 1, an amide bond may be formed by reaction between the primary amino group present on the amyloid-binding moiety and the anhydride function on the metal-chelating moiety. A direct linkage may also be formed by using an activating agent, such as a carbodiimide, to bind, for example, the primary amino group present on one moiety to the carboxy group present on the other moiety. A wide range of activating agents are known in the art and are suitable for use in the present invention.

In other preferred embodiments, the metal-chelating and amyloid-binding moieties are indirectly covalently linked to each other via a linker group. This can be accomplished by using any number of stable bifunctional agents well known in the art, including homofunctional and heterofunctional linkers (see, for example, Pierce Catalog and Handbook, 1994). The use of a bifunctional linker differs from the use of an activating agent in that the former results in a linking moiety being present in the inventive bifunctional molecule after reaction, whereas the latter results in a direct coupling between the two moieties involved in the reaction. The main role of the bifunctional linker is to allow the reaction between two otherwise chemically inert moieties. However, the bifunctional linker, which becomes part of the reaction product, can also be selected such that it confers some degree of conformational flexibility to the bifunctional molecule (e.g., the bifunctional linker comprises a straight alkyl chain containing several atoms, for example the straight alkyl chain contains between 2 and 10 carbon atoms).

A wide range of suitable homofunctional and heterofunctional linkers known in the art can be used in the context of the present invention. Preferred linkers include, but are not limited to, alkyl and aryl groups, including straight chain and branched alkyl groups, substituted alkyl and aryl groups, heteroalkyl and heteroaryl groups, that have reactive chemical functionalities such as amino, anhydride, hydroxyl, carboxyl, carbonyl groups, and the like.

As can readily be appreciated by those skilled in the art, a bifunctional molecule of the invention can comprise any number of amyloid-binding moieties and any number of metal-chelating moieties, linked to one another by any number of different ways. The amyloid-binding moieties within an inventive bifunctional molecule can be all identical or different. Similarly, the metal-chelating moieties within an inventive bifunctional molecule may be all identical or different. The precise design of a bifunctional therapeutic molecule will be influenced by its intended purpose(s) and the properties that are desirable in the particular context of its use.

II. Targeted Contrast Imaging Agents

Another aspect of the invention relates to a new class of targeted contrast imaging agents.

As already mentioned above, the diagnosis of amyloidosis currently involves histopathology of biopsies or tissue samples. The presence of amyloid is typically determined by the apple-green birefringence detected under crossed polarized light after staining with Congo red. However, biopsy of an affected organ is not free of complications and cannot satisfactorily reveal the extent or distribution of amyloid deposits (C. Friman and T. Pettersson, Curr. Opin. Rheumatol. 1996, 8: 6-71). In the case of Alzheimer's disease, amyloid deposition can only be assessed after death. This constitutes a major impediment to the study of the disease as well as to the development of more effective therapeutic methods.

An ideal probe for the diagnosis of amyloidosis would be one that has a high affinity and specificity for amyloid, exhibits a low toxicity and allows the detection, localization, and quantification of amyloid deposits in a patient. For the diagnosis of Alzheimer's disease and other neurodegenerative disorders associated with aggregation and accumulation of amyloid (or amyloid-like) proteins in the brain, an ideal probe should also be blood-brain barrier permeable and allow the non-invasive detection, localization, and quantification of amyloid deposits in the brain of live patients.

The present invention is directed to targeted, detectable reagents that meet some of the criteria listed above. Accordingly, the present invention provides targeted contrast imaging agents that are designed to (1) have some degree of attraction for amyloid, and (2) be detectable by imaging techniques. More specifically, the invention provides contrast imaging agents comprising at least one imaging moiety associated with at least one amyloid-binding moiety.

Amyloid-Binding Moieties

Amyloid-binding moieties in the contrast imaging agents of the invention play the same role than in the bifunctional therapeutic molecules described above: they are targeting entities that display some degree of attraction for amyloid, i.e., they specifically and/or efficiently interact with, bind to, or label amyloid deposits. Suitable amyloid-binding moieties for use in the design and development of contrast imaging agents are therefore identical to those listed above for the bifunctional therapeutic molecules.

In certain preferred embodiments, amyloid-binding moieties in inventive contrast imaging agents exhibit a high affinity and specificity for amyloid deposits. In other preferred embodiments, amyloid-binding moieties have a high affinity and specificity for Aβ amyloid. In yet other preferred embodiments, amyloid-binding moieties are capable of crossing the blood-brain barrier, which is, as noted above, an important property when the contrast imaging agent is intended to be used as an in vivo biological marker of amyloid deposits localized in the brain.

The potential carcinogenicity of certain azo dyes mentioned above, has little implication in the case of amyloid imaging studies since only a very minute, negligible amount of the highly specific amyloid-binding moiety would at any time be in contact with intestinal bacteria.

Imaging Moieties

In the context of the present invention, imaging moieties are entities that are detectable by imaging techniques such as Magnetic Resonance Imaging (MRI), Magnetic Resonance Spectroscopy (MRS), Single Photon Emission Computed Tomography (SPECT) and Positron Emission Tomography (PET). Preferably, imaging moieties are stable, non-toxic entities that retain their properties under in vitro and in vivo conditions.

MRI Imaging Moieties. In certain preferred embodiments, the contrast imaging agents of the invention are designed to be detectable by Magnetic Resonance Imaging.

MRI has evolved into one of the most powerful non-invasive techniques in diagnostic clinical medicine and biomedical research (P. Caravan et al., Chem. Rev. 1999, 99: 2293-2352; W. Khun, Angew. Chem. Int. Ed. Engl. 1990, 29: 1-19; M. M. Huber et al., Bioconjug. Chem. 1998, 9: 242-249; R. A. Moats et al., Angew. Chem. Int. Ed. Engl. 1997, 36: 726-728; X. Yu et al., Mag. Res. Med. 2000, 44: 867-872). MRI is an application of Nuclear Magnetic Resonance (NMR), a well known analytical method used in chemistry, physics and molecular structural biology. MRI can generate three dimensional structural information in relatively short time spans and is widely used as a non-invasive diagnostic tool to identify potentially maleficent physiological anomalies, to observe blood flow or to determine the general status of a cardiovascular system (P. Caravan et al., Chem. Rev. 1999, 99: 2293-2352).

MRI has the advantage (over other high-quality imaging methods) of not relying on potentially harmful ionizing radiation (A. R. Johnson et al., Inorg. Chem. 2000, 39: 2652-2660). In MRI, a contrast image of a biological sample or patient's body is provided by monitoring local variations in water concentrations and T₁ (spin-lattice) and T₂ (spin-spin) relaxation times of NMR signals from water protons (¹H). Often the clarity of MRI can be improved through the use of contrast imaging agents. Due to their paramagnetic properties, these agents decrease the T₁ and T₂ relaxation times by using their unpaired electrons to facilitate spin transfer. This results in an increase of concentration-dependent contrast and consequently an enhanced differentiation between anatomical structures.

Since the paramagnetic susceptibility of an entity (and hence its ability to shorten the T₁ and T₂ relaxation times of the proton nuclei of nearby water molecules) increases with the number of unpaired electrons (F. A. Cotton et al., “Basic Inorganic Chemistry”, John Wiley & Sons, New York, 1995, p. 68), ideal paramagnetic metal ions for use in MRI should, in principle, have as many unpaired electrons as possible. However, the complex of such paramagnetic metal ions with water molecules is highly toxic and therefore useless for in vivo imaging (W. Kuhn, Angew. Chem. Int. Ed. Engl. 1990, 29: 1-19). By complexing a paramagnetic metal ion to a ligand or metal-chelating moiety and leaving only one coordination site open for a water molecule, the toxicity has been found to be strongly reduced. Therefore, most MRI contrast agents typically consist of chelated paramagnetic metal ions.

Accordingly, in certain embodiments of the invention, the MRI contrast imaging agents are preferably designed such that the imaging moiety comprises at least one metal-chelating moiety complexed to a paramagnetic metal ion.

Suitable paramagnetic metal ions for use in the present invention include any of the paramagnetic metal ions known to be physiologically acceptable, good contrast enhancers in MRI, and easily incorporated into metal-chelating moieties. Preferably, the paramagnetic metal ion is selected from the group consisting of gadolinium III (Gd³⁺), chromium III (Cr³⁺), dysprosium III (Dy³⁺), iron III (Fe³⁺), manganese II (Mn²⁺), and ytterbium III (Yb³⁺). More preferably, the paramagnetic metal ion is gadolinium III (Gd³⁺). Gadolinium is an FDA-approved contrast agent for MRI, which accumulates in abnormal tissues causing these abnormal areas to become very bright (enhanced) on the MRI. Gadolinium is known to provide great contrast between normal and abnormal tissues in different areas of the body, in particular in the brain.

Suitable metal-chelating moieties for use in the present invention include any of the entities known in the art to complex paramagnetic metal ions detectable by MRI. Preferably, a metal-chelating moiety is a stable, non-toxic entity that binds a paramagnetic metal ion in such a way that it leaves one coordination site open for a water molecule and with such high affinity that, once complexed, the paramagnetic metal ion cannot be displaced by water.

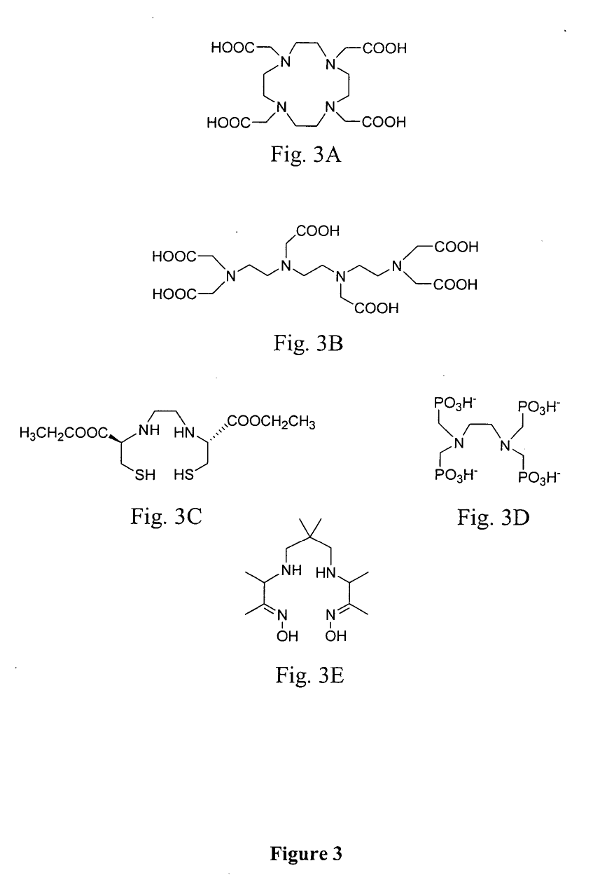

A number of such metal-chelating moieties have been used for the complexation of Gd³⁺. These include DTPA (FIG. 2G); 1,4,7,10-tetraazacyclododecane-N,N′,N″,N′″-tetraacetic acid (DOTA, whose chemical structure is presented in FIG. 3A); and derivatives thereof (see, for example, U.S. Pat. Nos. 4,885,363; 5,087,440; 5,155,215; 5,188,816; 5,219,553; 5,262,532; and 5,358,704; and D. Meyer et al., Invest. Radiol. 1990, 25: S53-55). However, gadolinium complexes of these ligands are salts under physiological conditions, and the requirement of nonparamagnetic cationic counterions increases the osmolality of the solution. A neutral gadolinium complex that retains high water solubility and relaxativity, has been prepared using DTPA-bis(amide) derivatives (U.S. Pat. No. 4,687,659).

Other metal-chelating moieties that complex paramagnetic metal ions include acyclic entities such as aminopolycarboxylic acids and phosphorus oxyacid analogues thereof (e.g., triethylenetetraminehexaacetic acid or TTHA, whose chemical structure is presented in FIG. 3B, and dipyridoxal diphosphate, DPDP, depicted on FIG. 3C) and macrocyclic entities (e.g., 1,4,7,10-tetraazacyclododecane-N,N′,N″-triacetic acid or DO3A, whose chemical structure is presented in FIG. 3D). Metal-chelating moieties may also be any of the entities described in U.S. Pat. Nos. 5,410,043; 5,277,895; and 6,150,376; or in F. H. Arnold, Biotechnol. 1991, 9: 151-156.

The synthesis of a family of novel MRI contrast imaging agents developed by insertion of Gd³⁺ in therapeutic bifunctional molecules of the invention is described in Example 2. The properties and uses of the inventive MRI imaging agents are reported in Examples 7 and 8, respectively.

MRS Imaging Moieties. In certain embodiments, the contrast imaging agents of the invention are designed to be useful in Magnetic Resonance Spectroscopy (MRS). More specifically, the present invention also provides contrast imaging agents comprising at least one metal-chelating moiety associated with at least one amyloid-binding moiety labeled with a stable paramagnetic isotope. Preferred stable paramagnetic isotopes are carbon-13 (¹³C) and fluorine-19 (¹⁹F).

Radioactive Imaging Moieties. In other preferred embodiments, the contrast imaging agents of the invention are designed to be detectable by Single Photon Emission Computed Tomography (SPECT) or Positron Emission Tomography (PET).

SPECT and PET are nuclear medicine imaging techniques which have been used to detect tumors, aneurysms (weak spots in blood vessel walls), irregular or inadequate blood flow to various tissues, blood cell disorders, and inadequate functioning of organs, such as thyroid and pulmonary function deficiencies. Both techniques acquire information on the concentration of radionuclides introduced into a biological sample or a patient's body. PET generates images by detecting the radiation emitted from short-lived radioactive substances, which are formed by bombarding non-radioactive chemicals with neutrons to create radioactive isotopes. PET detects the gamma rays given off at the site where a positron emitted from the radioactive substance collides with an electron in the tissue. A PET analysis results in a series of thin slice images of the body over the region of interest (e.g., brain, breast, liver). These thin slice images can be assembled into a three dimensional representation of the examined area. However, there are only few PET centers because they must be located near a particle accelerator device that is required to produce the short-lived radioisotopes used in the technique. SPECT is similar to PET, but the radioactive substances used in SPECT (e.g., ^{99m}TC, ¹²³I, ¹³³Xe) have longer decay times than those used in PET and emit single instead of double gamma rays. Although SPECT images exhibit less sensitivity and are less detailed than PET images, the SPECT technique exhibits several advantages over PET in that it does not require the proximity of a particle accelerator and is much less expensive than PET.

Accordingly, in certain preferred embodiments, the contrast imaging agents of the invention are designed to be detectable by Single Photon Emission Computed Tomography (SPECT). Preferably, the imaging moiety in the contrast imaging agent comprises at least one metal-chelating moiety complexed to a metal entity that is detectable by SPECT.

Suitable metal entities for use in the present invention are radionuclides known in the art to be physiologically acceptable, detectable by SPECT, and easily incorporated into metal-chelating moieties. Preferably, the radionuclide is selected from the group consisting of technetium-99m (^{99m}Tc), gallium-67 (⁶⁷Ga), yttrium-91 (⁹¹Y), indium-111 (¹¹¹In), rhenium-186 (¹⁸⁶Re), and thallium-201 (201Tl). Most preferably, the radionuclide is technetium-99m (^{99m}Tc). Over 85% of the routine nuclear medicine procedures that are currently performed use radiopharmaceutical methodologies based on ^{99m}Tc.

Suitable metal-chelating moieties for use in the present invention include any of the entities known to complex short-lived radionuclides detectable by SPECT. Preferably, metal-chelating moieties are stable, non-toxic entities that bind radionuclides detectable by SPECT with high affinity.

Metal-chelating moieties that complex radionuclides such as ^{99m}Tc are well known in the art (see, for example, “Technetium and Rhenium in Chemistry and Nuclear Medicine”, M. Nicolini et al., Eds., 1995, SGEditoriali: Padova, Italy). Suitable metal-chelating moieties include, for example, N₂S₂ and N₃S chelators (A. R. Fritzberg et al., J. Nucl. Med. 1982, 23: 592-598) which can complex a radionuclide through two nitrogen atoms and two sulfur atoms, or through three nitrogen atoms and one sulfur atom, respectively. Ethyl cysteine dimer (ECD, whose chemical structure is presented in FIG. 3C) is an N₂S₂ chelator well known in the art. N₂S₂ and N₃S chelators are, for example, described in U.S. Pat. Nos. 4,444,690; 4,670,545; 4,673,562; 4,897,255; 4,965,392; 4,980,147; 4,988,496; 5,021,556 and 5,075,099.

Other suitable metal-chelating moieties can be selected from polyphosphates (e.g., ethylenediaminetetramethylenetetraphosphonate, EDTMP, whose chemical structure is presented in FIG. 3D); aminocarboxylic acids (e.g., EDTA, N-(2-hydroxy)ethylenediamine-triacetic acid, nitrilotriacetic acid, N,N-di(2-hydroxyethyl)glycine, ethylenebis(hydroxyphenylglycine) and diethylenetriamine pentacetic acid); 1,3-diketones (e.g., acetylacetone, trifluoroacetylacetone, and thenoyltrifluoroacetone); hydroxycarboxylic acids (e.g., tartaric acid, citric acid, gluconic acid, and 5-sulfosalicyclic acid); polyamines (e.g., ethylenediamine, diethylenetriamine, triethylenetetraamine, and triaminotriethylamine); aminoalcohols (e.g., triethanolamine and N-(2-hydroxyethyl)ethylenediamine); aromatic heterocyclic bases (e.g., 2,2′-diimidazole, picoline amine, dipicoline amine and 1,10-phenanthroline); phenols (e.g., salicylaldehyde, disulfopyrocatechol, and chromotropic acid); aminophenols (e.g., 8-hydroxyquinoline and oximesulfonic acid); oximes (e.g., hexamethylpropyleneamine oxime, HMPAO, presented in FIG. 3E); Schiff bases (e.g., disalicylaldehyde 1,2-propylenediimine); tetrapyrroles (e.g., tetraphenylporphin and phthalocyanine); sulfur compounds (e.g., toluenedithiol, meso-2,3-dimercaptosuccinic acid, dimercaptopropanol, thioglycolic acid, potassium ethyl xanthate, sodium diethyldithiocarbamate, dithizone, diethyl dithiophosphoric acid, and thiourea); synthetic macrocyclic compounds (e.g., dibenzo[18]crown-6), or combinations of two or more of the above agents.

Preferred metal-chelating moieties are selected from the group consisting of polycarboxylic acids such as EDTA, DTPA, DOTA, DO3A; and derivatives, homologues and analogues thereof, or combinations thereof.

Other suitable metal-chelating moieties are described in U.S. Pat. No. 5,559,214, and in WO 95/26754, WO 94/09056, WO 94/29333, WO 94/08624, WO 94/08629, WO 94/13327, and WO 94/12216.

Preferably, the metal-chelating moieties contain at least one functional group that can be used (or is easily chemically converted to a different functional group that can be used) to covalently attach the metal-chelating moieties to amyloid-binding moieties. Suitable functional groups include, but are not limited to, amines (preferably primary amines), thiols, carboxy groups, and the like.

Synthesis of the Contrast Imaging Agents

The methods described above to prepare the inventive bifunctional molecules can be used to synthesize the contrast imaging agents.

Imaging moieties that comprise at least one metal-chelating moiety complexed to a metal entity can be prepared by any method known in the art. Complexation may be carried out before, during or after formation of direct or indirect covalent bonds between the metal-chelating and amyloid-binding moieties. Preferably, the complexation is carried out using an inventive bifunctional molecule as starting material (see Examples 2 and 3). When the metal entity is a short-lived radionuclide, the complexation is preferably carried out shortly before the contrast imaging agent is used.

Suitable complexation methods include, for example, direct incorporation of the metal entity into the metal-chelating moiety and transmetallation. When possible, direct incorporation is preferred. In this method, an aqueous solution of a metal-chelating moiety is generally exposed or mixed with a metal salt. The pH of the reaction mixture may be between about 4 and about 11. Preferably, the pH is between 5 and 9. More preferably, the reaction is carried out at a pH between 6 and 8. Direct incorporation methods are well known in the art and different procedures have been described (see, for example, WO 87/06229). Transmetallation is used when the metal entity needs to be reduced to a different oxidative state before incorporation. Transmetallation methods are well known in the art. Example 3 illustrates such a reaction, where the incorporation of ^{99m}Tc into a bifunctional molecule is carried out by reducing the metal ion to Tc(V) using SnCl₂.

As can be readily appreciated by those of ordinary skill in the art, a contrast imaging agent may comprise any number of amyloid-binding moieties and any number of imaging moieties, linked to one another by any number of different ways. The amyloid-binding moieties within a contrast imaging agent can be all identical or different. Similarly, the imaging moieties within a contrast imaging agent may be all identical or different. The design of a contrast imaging agent will be influenced by its intended purpose(s) and the properties that are desirable in the particular context of its use.

III. Uses of the Bifunctional Therapeutic Molecules

Another aspect of the present invention relates to systems for reducing or inhibiting amyloid toxicity. Accordingly, the present invention provides reagents and strategies for reducing or inhibiting the ability of amyloid (and amyloid-like) proteins to be toxic to their environment when aggregated in a β-sheet conformation. The present invention also provides reagents and strategies for reducing or inhibiting the amyloid-mediated generation of reactive oxygen species that have deleterious effects on a wide variety of biomolecules and can induce oxidative stress.

More specifically, the present invention provides targeted reagents that act as metal chelators and methods of using them for reducing or inhibiting amyloid toxicity in in vitro, in vivo and ex vivo systems as well as in living patients. The methods provided herein comprise using bifunctional molecules of the invention, which display a dual selectivity by both efficiently chelating transition metal ions, and exhibiting high affinity and specificity for amyloid deposits.

In certain preferred embodiments, the invention allows the reduction or inhibition of amyloid toxicity by preventing, slowing down, or stopping amyloid accumulation in the system or patient; and/or by promoting, inducing, or otherwise facilitating dissolution of amyloid deposits already present in the system or patient. This can be achieved when the metal-chelating moiety in the bifunctional molecule binds with high affinity at least one transition metal ion selected from the group consisting of zinc II (Zn²⁺), copper II (Cu²⁺), and iron III (Fe³⁺).

In other preferred embodiments, the invention allows the reduction or inhibition of amyloid toxicity by reducing, inhibiting, or otherwise interfering with amyloid-mediated production of reactive oxygen species. This can be achieved when the metal-chelating moiety in the bifunctional molecule binds with high affinity at least one redox active transition metal ion selected from the group consisting of copper II (Cu²⁺) and iron III (Fe³⁺).

As can be appreciated by those of ordinary skill in the art, a method involving the use of a bifunctional molecule with a metal-chelating moiety that binds with high affinity Cu²⁺ and/or Fe³⁺ can allow the reduction or inhibition of both the toxicity arising from the aggregated amyloid protein and that resulting from the generation of reactive oxygen species (since Cu²⁺ and Fe³⁺ are transition metal ions that can promote amyloid protein aggregation and redox active biometals that can be involved in the formation of reactive oxygen species).

More specifically, the present invention provides methods for reducing or inhibiting amyloid toxicity in a system, comprising contacting the system with an effective amount of a bifunctional molecule of the invention, or a pharmaceutical composition thereof.

The contacting may be carried out in vitro, in vivo, or ex vivo. For example, the contacting may be carried out by incubation.

The system may be any biological entity known to be able to produce and/or contain amyloid deposits. For example, the system may be a cell, a biological fluid, a biological tissue, or an animal. When the system is a cell, a biological fluid or a biological tissue, it may originate from a live patient (e.g., it may be obtained by biopsy) or a deceased patient (e.g., it may be obtained at autopsy). The patient may be a human or another mammal. In preferred embodiments, the cell, biological fluid, or biological tissue originates from a patient suspected to have a pathophysiological condition associated with amyloid accumulation. In other preferred embodiments, the cell, biological fluid, or biological tissue originates from a patient suspected to have a pathophysiological condition associated with accumulation of the amyloid-β peptide. In this particular case, the amyloid-binding moiety in the bifunctional molecule preferably exhibits a high affinity and specificity for Aβ amyloid.

The present invention also provides methods for preventing or treating a pathophysiological condition associated with amyloid accumulation in a patient. The methods described herein may be carried out (1) to delay or prevent the onset of the disease; or (2) to slow down or stop the progression, aggravation, or deterioration of the disease, or (3) to reverse or bring about ameliorations of the symptoms and signs of the disease; or (4) to cure the disease. The treatment may be administered prior to the onset of the disease, for a prophylactic or preventive action, or after initiation of the disease, for a therapeutic action.

More specifically, the present invention provides methods for treating a patient with a pathophysiological condition associated with amyloid accumulation, comprising administering to the patient an effective amount of a bifunctional molecule of the invention, or a pharmaceutical composition thereof.

Administration of the bifunctional molecule, or pharmaceutical composition thereof, may be performed by any suitable method known in the art, for example, by oral and parenteral administrations, including intravenous, intramuscular, and subcutaneous injections, and transdermal and enteral administrations.

The pathophysiological condition affecting the patient may be associated with accumulation of any amyloid or amyloid-like protein, such as the amyloid immunoglobulin light chain (AL); the serum amyloid associated protein (AA or SAP); the amyloid-β peptide (Aβ); the altered transthyretin (ATTR); the islet amyloid-polypeptide (IAPP or Amylin); the prion protein (PrP), and the like. The accumulation of the aggregated amyloid or amyloid-like protein may take place in any organ or tissue of the body and form fibrils, filaments, plaques, and/or tangles in the heart, brain, gastrointestinal system, liver, spleen, kidney, pancreas, lungs, joints, muscles, etc.

The pathophysiological condition may be any of the diseases and disorders known to be associated with amyloidosis. These include, but are not limited to, Type II diabetes mellitus, progressive supranuclear palsy, certain types of cancers of the endocrine system such as medullary carcinomas of the tyroid, familial amyloidosis (Finnish type); familial amyloid polyneuropathy (Portuguese type), familial amyloid polyneuropathy (Iowa type), familial amyloid cardiomyopathy (Danish type), familial amyloid nephropathy with urticaria and deafness (Muckle-Wells' syndrome), hereditary non-neuropathic systemic amyloidosis (Ostertag type), hereditary renal amyloidosis, myeloma or macroglobulinemia-associated idopathy associated with amyloid, systemic senile amyloidosis, Hodgkin's disease, Islets of Langerhans, isolated atrial amyloid, and familial Mediterranean fever.

In certain preferred embodiments, the inventive methods are directed to the prevention and treatment of pathophysiological conditions associated with amyloid and amyloid-like proteins that aggregate and accumulate preferentially in the brain. These pathophysiological conditions include, for example, the Prion diseases, that can affect humans (e.g., Creutzfeld-Jakob disease, Gerstmann-Straussler-Scheinker syndrome, Fatal Familial Insomnia, and Kuru diseases) as well as other mammals (e.g., bovine spongiform encephalopathy in cattle, scrapie in sheep, transmissible encephalopathy in mink, and chronic wasting disease in muledeer and elk); amyloidoses sometimes observed in the brain of individuals with head trauma; and neurodegenerative diseases such as Alzheimer's disease, Lewy body dementia, hereditary cerebral hemorrhage with amyloidosis (Dutch type and Icelandic type), Guam Parkinson-Dementia, and the form of Alzheimer's disease that affects adult Down's syndrome patients. In these cases, the amyloid-binding moiety in the inventive bifunctional molecule is selected such that it has the capability of crossing the blood-brain barrier.

IV. Detection Methods

In another aspect, the present invention allows the diagnosis of pathophysiological conditions associated with amyloid accumulation. In particular, the present invention allows the non-invasive diagnosis of neurodegenerative diseases characterized by the aggregation and accumulation of amyloid proteins in the brain of the patient. Accordingly, the invention provides reagents and strategies to detect the presence of amyloid deposits. More specifically, the invention provides targeted reagents that are detectable by imaging techniques and methods that allow the detection, localization and quantification of amyloid deposits in in vitro, in vivo, and ex vivo systems as well as in living patients. The methods provided herein are based on the use of inventive contrast imaging agents, which comprise at least one amyloid-binding moiety having a high affinity and specificity for amyloid deposits, associated with at least one imaging moiety that is detectable by imaging techniques. Alternatively, the methods provided herein may involve the use of inventive contrast imaging agents comprising at least one metal-chelating moiety associated with at least one amyloid-binding moiety labeled with a stable paramagnetic isotope.

More specifically, the present invention provides methods for detecting the presence of amyloid deposits in a system comprising the step of contacting the system with an imaging effective amount of a contrast imaging agent of the invention, or a pharmaceutical composition thereof. The contacting is preferably carried out under conditions that allow the contrast imaging agent to interact with an amyloid deposit present in the system so that the interaction results in the binding of the contrast imaging agent to the amyloid deposit. The contrast imaging agent that is bound to amyloid deposits present in the system is then detected using an imaging technique, and one or more images of at least part of the system are generated.

The contacting may be carried out by any suitable method known in the art. For example, the contacting may be carried out by incubation.

The system may be any biological entity known to be able to produce and/or contain amyloid deposits, for example, the system may be a cell, a biological fluid, a biological tissue, or an animal. When the system is a cell, a biological fluid or a biological tissue, it may originate from a live patient (e.g., it may be obtained by biopsy) or a deceased patient (e.g., it may be obtained at autopsy). The patient may be a human or another mammal.

In preferred embodiments, the cell, biological fluid, or biological tissue originates from a patient suspected to have a pathophysiological condition associated with amyloid accumulation. For example, the cell, biological fluid, or biological tissue may originate from a patient suspected of having a pathophysiological condition associated with accumulation of the amyloid-β peptide. In this particular case, the amyloid-binding moiety in the contrast imaging agent is selected for its high affinity and specificity for Aβ amyloid. In other preferred embodiments, the cell, biological fluid, or biological tissue has been contacted (in vitro or ex vivo) with a potential therapeutic agent for the treatment of a pathophysiological condition associated with amyloid accumulation.

In an aspect of the present invention, the method described above is used for identifying potential therapeutic agents. For example, images of at least part of a cell, biological fluid or biological tissue may be generated before and after contacting the cell, biological fluid or biological tissue with a potential therapeutic agent for the treatment of pathophysiological conditions associated with amyloid accumulation. Comparison of the “before” and “after” images allows the determination of the effects of the agent on the amyloid deposits present in the system. The invention also includes the therapeutic agents identified by this method.

The present invention also provides methods for detecting the presence of amyloid deposits in a patient. The methods comprise administering to the patient an imaging effective amount of a targeted contrast imaging agent of the invention, or a pharmaceutical composition thereof. The administration is preferably carried out under conditions that allow the contrast imaging agent (1) to reach the area(s) of the patient's body that may contain amyloid deposits and (2) to interact with any amyloid deposit present so that the interaction results in the binding of the contrast imaging agent to the amyloid deposit(s). After administration of the contrast imaging agent and after sufficient time has elapsed for the interaction to take place (for example after between 30 minutes and 48 hours), the contrast imaging agent bound to amyloid deposits present in the patient is detected using an imaging technique, and one or more images of at least part of the body of the patient are generated.

In one embodiment, this method is used to localize amyloid deposits in a patient. By comparing the results obtained from a patient suspected to have a pathophysiological condition associated with amyloid accumulation and images obtained from studies of clinically healthy individuals, the presence and distribution of amyloid deposits can be determined, and the diagnosis of amyloidosis confirmed. For example, the method can be used to localize amyloid plaques in the brain of a patient. In this case, the amyloid-binding moiety in the contrast imaging agent is selected such that it is capable of crossing the blood-brain barrier. In particular, the method can be used to detect and localize amyloid plaques in the brain of a patient suspected to have Alzheimer's disease. In this case, the amyloid-binding moiety in the contrast imaging agent has a high affinity and specificity for Aβ amyloid and is blood-brain barrier permeable. For brain imaging, the amount of the bound contrast imaging agent is measured and compared (as a ratio) to the amount of contrast imaging agent bound to the cerebellum of the patient. This ratio is then compared to the same ratio in the brains of age-matched clinically healthy patients.

The administration of the contrast imaging agent, or pharmaceutical composition thereof, can be carried out by any suitable method known in the art such as administration by oral and parenteral methods, including intravenous, intraarterial, intrathecal, intradermal, and intracavitory administrations, and enteral methods.

In preferred embodiments, the methods provided herein to detect the presence of amyloid deposits in a system or patient are carried out by using a contrast imaging agent of the invention, wherein the metal-chelating moiety is complexed to a paramagnetic metal ion as described above. The detection of amyloid deposits is then performed by Magnetic Resonance Imaging (MRI), and MR images are generated. Preferably, the paramagnetic metal ion is gadolinium III (Gd³⁺).

In other preferred embodiments, the detection methods are carried out by using a contrast imaging agent of the invention, wherein the metal-chelating moiety is complexed to a radionuclide as described above. The detection of amyloid deposits is then performed by Single Photon Emission Computed Tomography (SPECT), and SPECT images are generated. Preferably, the radionuclide is technetium-99m (^{99m}Tc).

In still other preferred embodiments, the detection methods are carried out by using a contrast imaging agent of the invention, wherein the amyloid-binding moiety is labeled with a stable paramagnetic isotope as described above. The detection of amyloid deposits is then performed by Magnetic Resonance Spectroscopy (MRS), and MR images are generated. Preferably, the stable paramagnetic isotope is carbon-13 (¹³C) or fluorine-19 (¹⁹F).

The methods of the invention that provide for detecting the presence of amyloid deposits in a patient or in a system can be used to diagnose a pathophysiological condition associated with amyloid accumulation. The diagnosis can be achieved by examining and imaging parts or the whole body of the patient or by examining and imaging a biological system (such as one or more samples of biological fluid or biological tissue) obtained from the patient. One or the other method, or a combination of both, will be selected depending on the nature of the clinical condition suspected to affect the patient. Comparison of the results obtained from the patient with data from studies of clinically healthy individuals will allow determination and confirmation of the diagnosis.

These methods can also be used to follow the progression of a pathophysiological condition associated with amyloidosis. For example, this can be achieved by repeating the method over a period of time in order to establish a time course for the presence, localization, distribution, and quantification of amyloid deposits in a patient.

These methods can also be used to monitor the response of a patient to a treatment for a pathophysiological condition associated with amyloid accumulation. For example, an image of part of the body of the patient that contains amyloid deposits (or an image of part of a cell, biological fluid, or biological tissue originating from the patient and containing amyloid deposits) is generated before and after submitting the patient to a treatment. Comparison of the “before” and “after” images allows to determine the effects of the treatment on the amyloid deposits and therefore to monitor the response of the patient to that particular treatment.

Pathophysiological conditions that may be diagnosed, or whose progression can be followed by the methods provided herein may be associated with accumulation of any amyloid or amyloid-like protein, as enumerated above. Aggregated amyloid or amyloid-like proteins may accumulate in any organ or tissue of the body and form fibrils, filaments, plaques, and/or tangles. Organs such as the heart, brain, gastrointestinal system, liver, spleen, kidney, pancreas, lungs, joints, muscles and the like may be examined and imaged using the inventive methods provided herein

Pathophysiological conditions that may be diagnosed, or whose progression can be followed by the inventive methods provided herein may be any of the diseases and disorders known to be associated with amyloidosis. For example, the inventive methods may be used to diagnose conditions such as Type II diabetes mellitus, progressive supranuclear palsy, certain types of cancers of the endocrine system such as medullary carcinomas of the tyroid, familial amyloidosis (Finnish type); familial amyloid polyneuropathy (Portuguese type), familial amyloid polyneuropathy (Iowa type), familial amyloid cardiomyopathy (Danish type), familial amyloid nephropathy with urticaria and deafness (Muckle-Wells' syndrome), hereditary non-neuropathic systemic amyloidosis (Ostertag type), hereditary renal amyloidosis, myeloma or macroglobulinemia-associated idopathy associated with amyloid, systemic senile amyloidosis, Hodgkin's disease, Islets of Langerhans, isolated atrial amyloid, and familial Mediterranean fever.

In certain preferred embodiments, the inventive methods are directed to the diagnosis of pathophysiological conditions associated with amyloid and amyloid-like proteins that aggregate and accumulate preferentially in the brain. These pathophysiological conditions include, for example, the Prion diseases, that can affect humans (e.g., Creutzfeld-Jakob disease, Gerstmann-Straussler-Scheinker syndrome, Fatal Familial Insomnia, and Kuru diseases) as well as other mammals (e.g., bovine spongiform encephalopathy in cattle, scrapie in sheep, transmissible encephalopathy in mink, and chronic wasting disease in muledeer and elk); amyloidoses sometimes observed in the brain of individuals with head trauma; and neurodegenerative diseases such as Alzheimer's disease, Lewy body dementia, hereditary cerebral hemorrhage with amyloidosis (Dutch type and Icelandic type), Guam Parkinson-Dementia, and the form of Alzheimer's disease that affects adult Down's syndrome patients. In these cases, the amyloid-binding moiety in the inventive contrast imaging agent is selected such that it has the capability of crossing the blood-brain barrier.

V. Formulation, Dosage and Administration

Bifunctional Therapeutic Molecules

The bifunctional therapeutic molecules described herein may be administered per se or in the form of a pharmaceutical composition. Accordingly, the present invention provides pharmaceutical compositions comprising an effective amount of at least one bifunctional molecule, or a physiologically tolerable salt thereof, and at least one pharmaceutically acceptable carrier. The specific formulation will depend upon the route of administration selected. Bifunctional therapeutic molecules, or pharmaceutical compositions thereof, may be administered by any suitable method known in the art. Examples of suitable routes include oral and parenteral administrations, including intravenous, intramuscular, intraperitoneal, and subcutaneous injections, transdermal and enteral administrations, and the like.

Pharmaceutical compositions for oral administration may be obtained by combining a bifunctional molecule of the invention with one or more pharmaceutically acceptable carriers or diluents. The use of such carriers allows the bifunctional molecules of the invention to be formulated, for example, as tablets, capsules, pills, dragees, liquids, gels, syrups, slurries, and suspensions. Pharmaceutically acceptable carriers and diluents for oral administration are well known in the art (see, for example, Remington's Pharmaceutical Sciences, 1990), and include any and all solvents, dispersion media, antibacterial and antifungal agents, isotonic and absorption delaying agents, and the like. Such pharmaceutical compositions should contain at least 1% by weight of active compound. The percentage of the compositions may be varied and may conveniently be between about 5 to about 80% of the weight of the unit. The amount of active compound in therapeutically useful compositions is such that a suitable dosage will be obtained. Preferred compositions according to the present invention are prepared so that an oral dosage unit form contains between about 0.5 μg and 2000 mg of active compound.

Oral formulations may optionally contain other conventional, non-toxic components such as fillers and binders (e.g., sugars such as lactose, sucrose, mannitol, and sorbitol; and cellulose preparations such as starch, gelatin, methyl cellulose, hydroxypropylmethylcellulose, sodium carboxymethylcellulose, and polyvinylpyrrolidone); excipients (e.g., dicalcium phosphate); disintegrating agents (e.g., cross-linked polyvinyl pyrrolidone, agar, alginic acid, and sodium alginate); lubricants (e.g., magnesium stearate); and flavoring agents (e.g., peppermint, oil of wintergreen, and cherry flavoring). When the formulation forms a capsule, it may contain, in addition to materials listed above, liquid or semi-liquid vehicles (e.g., fatty oils, liquid parrafin, and liquid polyethylene glycols). Various other materials may be present as coatings or to otherwise modify the physical form of the dosage unit. For example, tablets, pills, or capsules may be coated with shellac, sugar, or both. A syrup or elixir may contain the active compound, sucrose as a sweetening agent, methyl and propylparabens as preservatives, a dye, and flavoring such as cherry or orange flavor. Any material used in the preparation of oral pharmaceutical compositions should be pharmaceutically pure and substantially non-toxic in the amounts employed.

The bifunctional molecules of the invention may also be formulated for parenteral administration by injection (e.g., by bolus injection or continuous infusion), and presented in unit dosage form (e.g., in ampoules or in multi-dose containers). Dosage unit forms for injection are especially advantageous for ease of administration and uniformity of dosage. The term “Dosage unit form”, as used herein, refers to a physically discrete unit suited as unitary dosage for the patient to be treated. Each unit contains a predetermined quantity of active material calculated to produce the desired therapeutic effect. The dosage unit forms directly depend on the characteristics of the active compound and the desired therapeutic effect. For example, a unit dosage form contains the principal active compound in amounts ranging from 0.5 μg to about 2000 mg. Alternatively, amounts ranging from 200 ng/kg of body weight to above 10 mg/kg of body weight may be administered. The amounts may be for individual active compounds or for the combined total of active compounds.

Parenteral compositions may be suspensions, emulsions, or aqueous and non-aqueous solutions of the active bifunctional molecule, and may optionally contain other auxiliaries such as suspending, stabilizing, and/or dispersing agents. Lipophilic solvents or vehicles (e.g., fatty oils, synthetic fatty acid esters, and liposomes) can be used to prepare suspensions and emulsions. The viscosity of aqueous parenteral formulations may be increased by adding substances such as sodium carboxymethylcellulose, sorbitol, and dextran.

Alternatively, the bifunctional molecules of the invention may be formulated to allow a controlled delivery of the active ingredient. Control release compositions are well known in the art (see, for example, Remington's Pharmaceutical Sciences, 1990) and may take the form of microcapsules, suppositories, or depot preparations. These pharmaceutical compositions may be obtained by incorporating or entrapping the active molecule(s) into particles of polymeric material (such as, for example, polyesters, polyamino acids, polyvinyl pyrrolidone, hydrogels, polylactic acid, ethylene vinylacetate, methylcellulose, hydroxymethylcellulose, and carboxy-methylcellulose) or in colloidal drug delivery systems (such as, for example, liposomes, microspheres, micro- or macro-emulsions, nanoparticles, and nanocapsules). Depot preparations may be administered by implantation or transcutaneous delivery, intramuscular injection, or through the use of a transdermal patch (see, for example, the devices described in U.S. Pat. Nos. 4,708,716 and 5,372,579).

The bifunctional therapeutic molecules of the invention, or pharmaceutical compositions thereof, may be administered singly, in combination with other reagents of the invention, and/or combined with other therapeutic agents, the nature of which will depend, in part, on the condition being treated. For example, in the case of Alzheimer's disease, the bifunctional molecules of the invention can be administered in combination with FDA-approved therapeutics, such as donepezil hydrochloride (Aricept®), tacrine (Cognex®), rivastigmine (Exelon®), and velnacrine (Mentane®), which are acetylcholinesterase inhibitors that act as cognitive enhancers and are known to provide slight relief in some AD patients. The ability to determine combinations of compounds suitable to treat particular disorders is well within the capabilities of trained physicians.

The bifunctional molecules of the invention, or pharmaceutical compositions thereof, can be administered therapeutically to treat a variety of pathophysiological conditions associated with amyloid accumulation, (i.e., after the onset of the disease) or prophylactically to prevent these pathophysiological conditions.

Administration of the bifunctional molecules of the invention, or pharmaceutical compositions thereof, will be in a dosage such that the amount delivered is effective for its intended purpose. The route of administration, formulation and dosage administered will be dependent upon the age, sex, weight and health condition of the patient; the particular pathophysiological condition to be treated (systemic or localized, primary or secondary amyloidosis); the extent of the disease; the potency, bioavailability, in-vivo half-life and severity of the side effects of the bifunctional therapeutic molecule. These factors are readily determinable in the course of therapy. Alternatively or additionally, the dosage to be administered can be determined from studies using animal models for the particular condition being treated, and/or from animal or human data obtained for compounds which are known to exhibit similar pharmacological activities. The total dose required for each treatment may be administered by multiple dose or in a single dose. Adjusting the dose to achieve maximal efficacy based on these or other methods are well known in the art and are within the capabilities of trained physicians.

Suitable patients with pathophysiological conditions associated with amyloid accumulation can be identified by laboratory tests and medical history. In particular, the presence, localization, distribution, and quantification of amyloid deposits can be determined by one of the inventive methods described herein that involve the use of targeted contrast imaging agents and imaging techniques.

Targeted Contrast Imaging Agents

The present invention also provides pharmaceutical compositions comprising targeted contrast imaging agents. More specifically, the pharmaceutical compositions of the invention comprise an imaging effective amount of at least one contrast imaging agent described above, or a physiologically tolerable salt thereof, and at least one pharmaceutically acceptable carrier. In certain preferred pharmaceutical compositions, the imaging moiety of the contrast imaging agent comprises at least one metal-chelating moiety complexed to a paramagnetic metal ion or to a radionuclide. Preferably, the paramagnetic metal ion is gadolinium III (Gd³⁺); the radionuclide is technetium-99m (^{99m}Tc). In other preferred embodiments, the amyloid-binding moiety in the contrast imaging agent is labeled to a stable paramagnetic isotope. Preferably, the stable paramagnetic isotope is carbon-13 (¹³C) or fluorine 19 (¹⁹F).

Administration of contrast imaging agents, or pharmaceutical compositions thereof, may be carried out by any suitable method known in the art, such as those described in Remington's Pharmaceutical Sciences. Depending on the particular type of amyloidosis suspected to affect the patient and the body site to be examined, the contrast imaging agent may be administered locally or systemically, and delivered orally (as solids, solutions, or suspensions) or by injection (for example, intravenously, intraarterially, intrathecally (i.e., via the spinal fluid), intradermally, or intracavitory).

For oral administration, the contrast imaging agents of the invention may be formulated as described above in the case of the bifunctional therapeutic molecules.

For administration by injection, pharmaceutical compositions of contrast imaging agents may be formulated as sterile aqueous or non-aqueous solutions or alternatively as sterile powders for the extemporaneous preparation of sterile injectable solutions. Such pharmaceutical compositions should be stable under the conditions of manufacture and storage, and must be preserved against the contaminating action of microorganisms such as bacteria and fungi.

Pharmaceutically acceptable carriers are solvents or dispersion media such as aqueous solutions (e.g., Hank's solution, alcoholic/aqueous solutions, or saline solutions), and non-aqueous carriers (e.g., propylene glycol, polyethylene glycol, vegetable oil and injectable organic esters such as ethyl oleate). Injectable pharmaceutical compositions may also contain parenteral vehicles (such as sodium chloride and Ringer's dextrose), and/or intravenous vehicles (such as fluid and nutrient replenishers); as well as other conventional, pharmaceutically acceptable, non-toxic excipients and additives including salts, buffers, and preservatives such as antibacterial and antifungal agents (e.g., parabens, chlorobutanol, phenol, sorbic acid, thirmerosal, and the like). Prolonged absorption of the injectable compositions can be brought about by adding agents that can delay absorption (e.g., aluminum monostearate and gelatin). The pH and concentration of the various components can readily be determined by those skilled in the art.

Sterile injectable solutions are prepared by incorporating the active compound(s) in the required amount of the appropriate solvent with various of the other ingredients enumerated above, followed by sterilization, for example, by filtration or irradiation. In the case of sterile powders for the preparation of sterile injectable solutions, the preferred methods of preparation are vacuum drying and the freeze-drying techniques.

Generally, the dosage of detectable contrast imaging agent will vary depending on considerations such as age, sex, and weight of the patient, as well as the particular pathophysiological condition suspected to affect the patient, the extent of the disease, and the area(s) of the body to be examined. Factors such as contraindications, concomitant therapies, and other variables are also to be taken into account to adjust the dosage of detectable contrast imaging agent to be administered. This can, however, be readily achieved by a trained physician.

In general, a suitable daily dose of a pharmaceutical composition of the invention corresponds to the lowest amount of contrast imaging agent that is sufficient to allow detection of any amyloid deposit present in the patient. To minimize this dose, it is preferred that administration be intravenous, intramuscular, intraperitoneal, or subcutaneous, and preferably proximal to the site to be examined. For example, intravenous administration is appropriate for imaging the urinary tract; intraspinal administration is better suited for imaging of the brain and central nervous system; while oral administration to an unfed patient is appropriate for imaging of the gastrointestinal tract.

The radioactive contrast imaging agents of the invention are preferably administered in the range of 0.1 to about 10 mCuries/kg of body weight per day. The paramagnetic contrast imaging agents of the invention are preferably administered in the range of 0.02 to 1.3 mmoles/kg of body weight per day.

### EXAMPLES

The following examples describe some of the preferred modes of making and practicing the present invention. However, it should be understood that these examples are for illustrative purposes only and are not meant to limit the scope of the invention.

Some of the results presented below have been reported in a scientific paper (A. Dedeoglu et al., Exp. Geront., 2004, 39: 1641-1649), which is incorporated herein by reference in its entirety.

### Example 1

### Synthesis of a Family of Amyloid-binding, Metal-Chelating Agents

A family of novel bifunctional molecules has been designed and is being developed. The new bifunctional molecules comprise one metal-chelating moiety directly, covalently linked to two identical amyloid-binding moieties. The metal-chelating moiety, which is common to all the bifunctional molecules of the family is diethylene triaminepentaacetic acid (DTPA), a metal chelator that is well-known in the art. As shown in FIG. 4A, the amyloid-binding moiety of the parent molecule of the family is a benzothioflavin derivative, which belongs to a family of thioflavin analogues that have been reported to be blood-brain barrier permeable in rodents and to exhibit a high affinity for Aβ amyloid (W. E. Klunk et al., Life Sci. 2001, 69: 1471-1484).

Other members of the family are analogues of the parent bifunctional molecule, in which the aromatic ring of the benzothiazole moiety is substituted with one or more than one functional group including, for example, 4-dimethylamino; 4-amino; 4-chloro; 4-chloro-5-ethyl; 4-acetyl; 5-carboxyl; 5-sulfonoxyl; 5-bromo; 4-, 5- or 6-methyl; 5-trifluoromethyl; 4-ethoxyl; 4-, 5- or 6-methylsulfonyl; and 4-, 5- or 6-hydroxyl (see FIG. 4B).

All the reagents and solvents used in the syntheses reported below were obtained from Aldrich Chemical (St. Louis, Mo.) Acros Organics (Pittsburg, Pa.), or Lancaster Synthesis Inc. (Windham, N.H.) at the highest purity grade available and used without prior purification.

The synthesis of the parent bifunctional molecule of the family (compound XH1) is described herein. As shown in the following scheme, the preparation of XH1 involves the formation of amide bonds between the metal-chelating moiety and the amyloid-binding moieties. The reaction was carried out according to a procedure adapted from previously published synthetic methods (W. E. Klunk et al., Life Sci. 2001, 69: 1471-1484; D. Shi et al, J. Med. Chem. 1996, 39: 3375-3384; and M. S. Konings et al., Inorg. Chem. 1990, 29: 1488-1491).

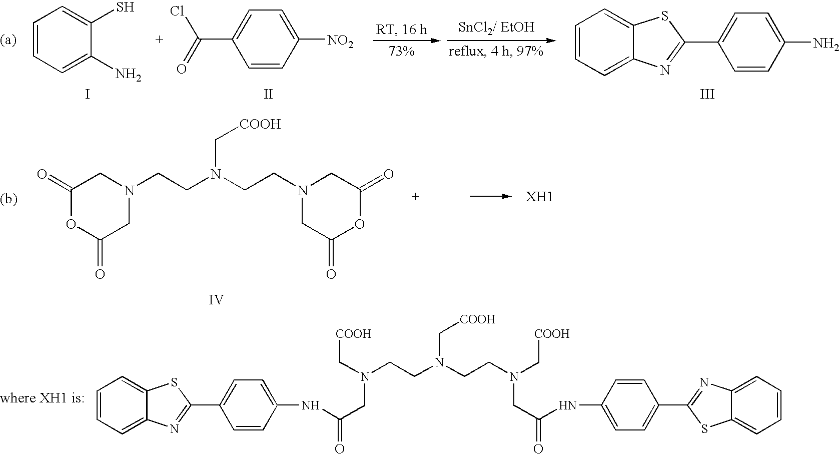

Step (a) of the synthesis gave the thioflavin analogue, 2-(4′-aminophenyl)-benzothiazole (compound III), which was prepared by reduction of the product of direct coupling between 4-nitrobenzoyl chloride (compound II) and 2-aminothiophenol (compound I). In step (b) of the synthesis, DTPA-bis(anhydride), compound IV, was reacted with an excess of the thioflavin analogue to form the desired compound XH1.

More precisely, compound I (10 g, 80 mmol) and compound II (15 g, 80 mmol) in anhydrous benzene (200 mL) were stirred at room temperature for 16 hours. Following extraction with ethyl acetate, the solvent was evaporated, and the residue was purified by flash chromatography (hexane:ethyl acetate; 85:15, v:v) to give 15 g (73%) of 2-(4′-nitrophenyl)-benzothiazole as a pale-yellow solid. A mixture of this intermediate (10 g, 40 mmol) and tin(II) chloride dihydrate (20 g, 90 mmol) in ethanol was then refluxed under nitrogen for 4 hours. Ethanol was removed by evaporation, and the residue was dissolved in ethyl acetate (200 mL). The resulting solution was washed with 1 M NaOH (3×200 mL) and with water (3×200 mL). Evaporation of the solvent gave 10 g (97%) of 2-(4′-aminophenyl)-benzothiazole.

DTPA-bis(anhydride) (2.5 g, 7.0 mmol) was then added in portions over 30 minutes to an ice-cold stirred solution of compound III (8.85 g, 7.8 mmol) in ethanol. After addition of water (150 mL), the resulting reaction mixture was stirred for another 12 hours at ambient temperature. The solution obtained by concentrating the reaction mixture under reduced pressure and adding water (500 mL) was adjusted to pH 2.5 with concentrated HCl to induce the formation of crystals. After collection, the crystals were recrystallized from ethanol to give pure XH1 (yield: 71%).

The parent bifunctional molecule obtained was characterized by LC/MS and

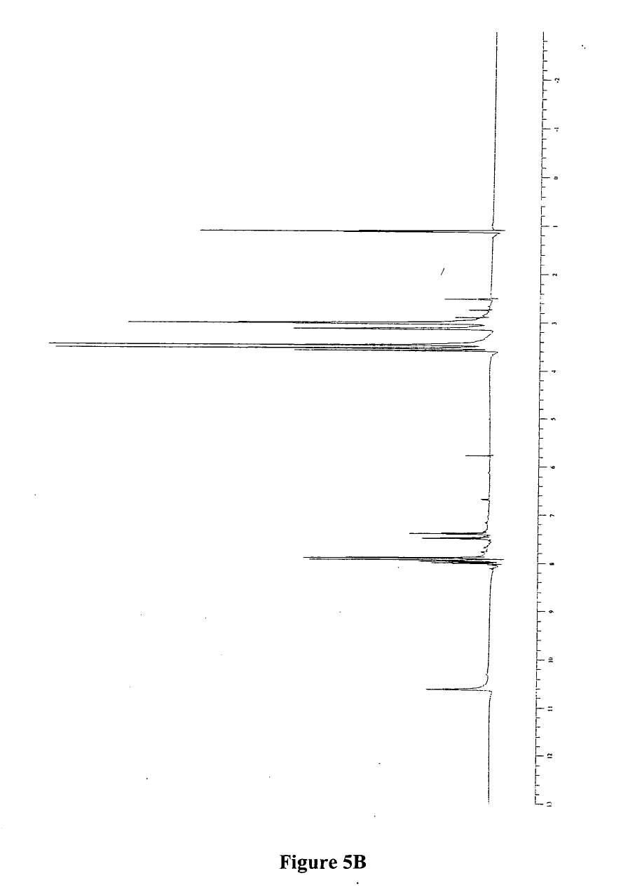

¹H-NMR. Proton-NMR spectra were recorded using a Bruker AVANT instrument operating at 250 MHz. Chemical shifts were reported in ppm using DMSO as reference. Reverse-phase LC/MS analyses were carried out by M-Scan, Inc. (West Chester, Pa.) via FAB-MS. LC-MS chromatography was performed on a Symmetry™ C18, 3.5 μM, 2.1×50 mm column (Waters Corporation, Milford, Mass.) using a flow rate of 1 mL/min. and a 10 min gradient of 15-85% acetonitrile in water, with a constant concentration of 0.1% formic acid. The mass spectrum and ¹H-NMR spectrum of XH1 are presented in FIG. 5.

The synthetic route described above can be used and adapted to the preparation of the other bifunctional molecules of the family.

### Example 2

### Synthesis of a Family of MRI Contrast Imaging Agents

Contrast imaging agents detectable by Magnetic Resonance Imaging (MRI) can be prepared from the bifunctional molecules described in Example 1. As shown below in the case of the parent compound, synthesis of the MRI contrast imaging agent Gd-XH1, involves insertion of gadolinium III (Gd³⁺) into XH1. This reaction was carried out according to a method previously reported (M. S. Konings et al., Inorg. Chem. 1990, 29: 1488-1491).

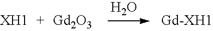

More specifically, a mixture of XH1 (12.1 g, 25.0 mmol) and gadolinium oxide, Gd₂O₃ (4.53 g, 12.4 mmol), in water (30 mL) was refluxed for 5 hours. Colorless crystals of the gadolinium complex were quantitatively formed by adjusting the pH of the solution to 6.5 with 1 M NaOH.

### Example 3

### Synthesis of a Family of SPECT Contrast Imaging Agents

Similarly, contrast imaging agents detectable by Single Photon Emission Computed Tomography (SPECT), can be prepared from the bifunctional molecules described in Example 1. The reaction, which is shown below in the case of the parent bifunctional molecule, is carried out by inserting technetium-99m using the stannous reduction at pH 6.5 procedure described in U.S. Pat. No. 4,434,151. This reaction yields the technetium complex, compound ^{99m}Tc-XH1, quantitatively.

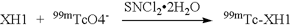

More specifically, XH1 (8.25 g, 0.17 mmol) is dissolved in 1.0 mL of ethanol and 1.0 M sodium acetate at pH 5.5, and 1.0 mL generator eluant of ^{99m}TcO₄₋ (5-50 mCi) in saline is added to the reaction mixture. Addition of 0.2 mL of a stannous solution, prepared by dissolving 2.0 mg of SnCl₂.H₂O per mL of ethanol, produces the technetium complex. After 15-30 minutes, the labeling efficiency can be determined by electrophoresis.

### Example 4

### Cell-Free Assays to Test the Biological Activity of Bifunctional Molecules

In the following paragraphs, various cell-free assays are described that can be used to test the biological activity of the inventive bifunctional molecules. More specifically, the first series of assays allow to assess the ability of a bifunctional molecule of interest to reduce, inhibit or otherwise interfere with the binding of redox active transition metal ions to the amyloid-β peptide leading to Aβ aggregation. The second series of assays allow to evaluate the ability of a bifunctional molecule of interest to inhibit the amyloid-mediated reduction of redox active transition metal ions. The third series of assays allow to assess the inhibiting effects of an inventive bifunctional molecule on metal- and amyloid-mediated production of reactive oxygen species, such as H₂O₂, O₂.⁻, and .OH. The last assay described in this section allows to evaluate the ability of a bifunctional molecule to dissolve metal-induced Aβ aggregates.

Inhibition of the Metal-Amyloid-β Peptide Binding

The binding of redox active transition metal ions to an amyloid protein is known to (1) promote the aggregation and accumulation of the protein, (2) reduce the transition metal ions, and (3) generate reactive oxygen species such as hydrogen peroxide (H₂O₂), superoxide radical anion (O₂.⁻) and hydroxyl radical (.OH). In the presence of an inventive bifunctional molecule, the efficiency of these three processes should be at least reduced, at best completely inhibited. The following cell-free assays can be used to evaluate the effects of bifunctional molecules of the invention on the binding of redox active transition metal ions to the amyloid-β peptide by assessing their effects on these three secondary processes.

(a) Synthesis of Aβ Peptides

Human Aβ peptides (Aβ₁₋₄₀ and Aβ₁₋₄₂) were synthesized using solid-phase Fmoc chemistry at the W. M. Keck Foundation Biotechnology Resource Laboratory (Yale University, New Haven, Conn.); Glabe Laboratory (University of California, Irvine, Calif.); or Multhaup Laboratory (University of Heidelberg, Germany); or purchased from U.S. Peptide, Inc. (Rancho Cucamonga, Calif.); or Aldrich-Sigma (St. Louis, Mo.). The syntheses were confirmed by purification by reverse-phase HPLC, amino acid sequencing, and mass spectrometry. Synthetic Aβ peptides were dissolved in trifluoroethanol (30% in Milli-Q water (Millipore Corporation, Milford, Mass.)) or 20 mM HEPES (pH 8.5) at a concentration of 0.5-1.0 g/mL, centrifuged for 20 minutes at 10,000×g and the supernatant (stock Aβ) used for subsequent assays on the day of the experiment.

The concentration of the stock solution of Aβ was determined by UV spectroscopy at 214 nm or by Micro BCA protein assay (Pierce, Rockford, Ill.). The Micro BCA assay was performed by adding 10 μL of stock Aβ (or bovine serum albumin (BSA) standard) to 140 μL of distilled water, and then adding an equal volume of the Micro BCA Protein Assay Reagent (150 μL) to a 96-well plate and measuring the absorbance at 562 nm. The concentration of Aβ was determined using the BSA standard curve. Prior to use, all buffers and stock solutions of metal ions (e.g., chloride salts) were filtered through a 0.22 μm filter (Gelan Sciences, Ann Arbor, Mich.) to remove any particulate matter.

Two different methods can be used to investigate the reversibility of Zn 2+-induced Aβ aggregation by XH1: turbidity and immuno-filtration.

(b) Inhibition of Metal-Induced Aggregation of Aβ measured by Turbidity

Turbidity measurements were performed as previously described (X. Huang et al., J. Biol. Chem. 1997, 272: 26464-26470). Aβ₁₋₄₀ (10 μM) was co-incubated with Zn²⁺ (25 μM) in presence or absence of 25 μM of compound XH1 (the parent bifunctional molecule) for 1 hour in 67 mM phosphate buffer, 150 mM NaCl (pH 7.4) and turbidity measurements were taken at four 1 min. intervals. Subsequently, 20 μL aliquots of 10 mM of the bifunctional molecule to be tested or 10 mM Zn²⁺ were added to the mixture alternatively, and following a 2 min. delay, a further four readings were taken at 1 min. intervals. After the final addition of the bifunctional molecule and turbidity reading, the mixtures were incubated for an additional 30 minutes before taking the last readings. For control purposes, the same experiment was carried out using diethylene triaminepentaacetic acid (DTPA) instead of compound XH1 (DTPA is both a well-known metal-chelator and the metal-chelating moiety comprised in XH1).

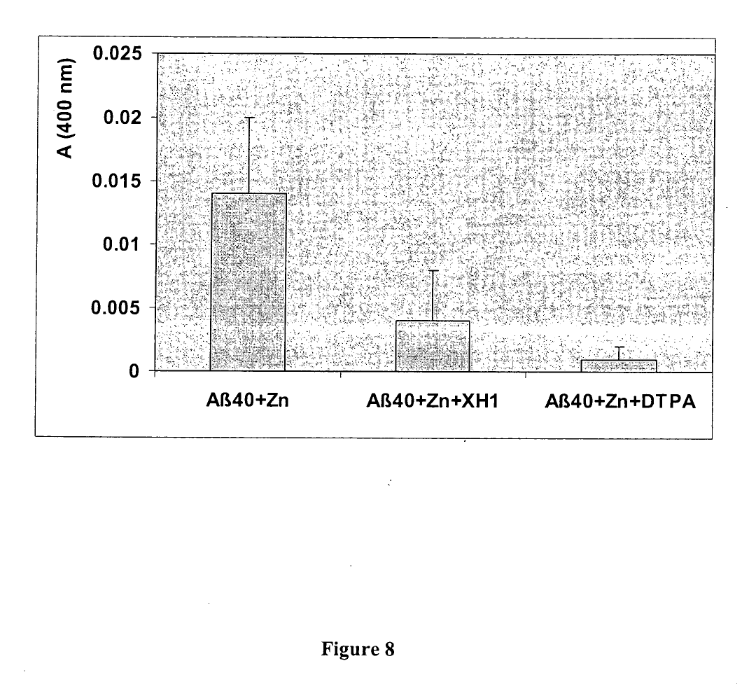

The results of these experiments, which are presented in FIG. 8, show that both DTPA and the inventive parent bifunctional molecule significantly decrease the Zn²⁺-induced aggregation of Aβ₁₋₄₀ in solution, with the strongest effect observed for DTPA.

(c) Inhibition of Metal-Induced Aggregation of Aβ measured by Immunofiltration

In a second method, that can be used for assessing the effects of bifunctional molecules of the invention on metal-induced Aβ aggregation, physiological concentrations of Aβ (8 nM) are added to mixtures containing 150 mM NaCl, 20 mM HEPES (pH 7.4), 100 nM BSA with ZnCl₂ (0, 0.1, 0.2, 0.5 and 2 μM) and incubated (30 min., 37° C.) in the presence or absence of the bifunctional molecule to be tested (1-5 μM). The reaction mixtures (200 μL) are then placed into the 96-well Easy-Titer ELISA system (Pierce, Rockford, Ill.) and filtered through a 0.22 μm cellulose acetate filter (MSI, Westboro, Mass.). Aggregated particles are fixed to the membrane (0.1% glutaraldehyde, 15 min.), washed thoroughly and then probed with the anti-Aβ monoclonal antibody 6E10 (Senetek, Maryland Leights, Mich.). Blots are washed and exposed to film in the presence of ECL chemiluminescence reagents (Amersham Biosciences Corp., Piscataway, N.J.). Immunoreactivity is then quantified by transmittance analysis of ECL film from the immunoblots.

Inhibition of the Amyloid-mediated Reduction of Redox Active Transition Metal Ions

The metal reduction assay can be performed using a 96-well microtiter plate (Corning Costar, Acton, Mass.) according to a method based on a modification of established protocols (J. W. Landers et al., Amer. Clin. Path. 1958, 29: 590-592). Aβ peptides (10 μM) or Vitamin C (100 μM), metal ions (10 μM, Fe(NO₃)₃ or Cu(NO₃)₂), and reduced metal ion indicators, bathophenanthrolinedisulfonic acid (BP, for Fe²⁺, 200 μM, Aldrich-Sigma) or bathocuproinedisulfonic acid (BC, for Cu⁺, 200 μM, Aldrich-Sigma), are co-incubated in phosphate buffered saline (PBS), pH 7.4, for 1 hour at 37° C. in the presence or absence of the inventive bifunctional molecule to be tested (200 μM). The metal ion solutions are prepared by direct dilution in the buffer from their aqueous stocks purchased from the National Institute of Standards and Technology (NIST). Absorbances are then measured at 536 nm (Fe²⁺-BP complex) and 483 nm (Cu⁺-BC complex), respectively, using a 96-well plate reader (SPECTRAmax 250, Molecular Devices, C A). Absorbances of control samples are also measured to estimate the contribution of light scattering and determine the background buffer signal at these wavelengths. The net absorbances (ΔA) at 536 nm or 483 nm are obtained by deducting the absorbances from these controls from the absorbances generated by the peptide and metal in the presence of the respective indicator and bifunctional molecule.

The concentrations of reduced metal ions (Fe²⁺ or Cu⁺) are quantified based on the formula: [Fe²⁺] or [Cu⁺]=(ΔA×10⁶)/(ε×l), where l is the measured equivalent vertical pathlength for a well of 300 μL volume as described in the instrument's specifications manual (0.856 cm for Fe²⁺; 1.049 cm for Cu⁺); ε is the known molecular absorbance for the complex, i.e., 7124 M⁻¹cm⁻¹ for Fe²⁺-BP and 2762 M⁻¹cm⁻¹ for Cu⁺-BC.

Inhibition of the Metal- and Amyloid-Mediated Production of Reactive Oxygen Species

H₂O₂ Assay. The H₂O₂ assay can be performed in a UV-transparent 96-well microtiter plate (Molecular Devices, C A), according to a procedure adapted from existing protocols (J. C. Han et al., Anal. Biochem. 1996, 234: 107-109; and J. C. Han et al., Biochem. 1994, 220: 5-10). The Aβ peptide (Aβ₁₋₄₂ or Aβ₁₋₄₀; 10 μM) or Vitamin C (10 μM), Fe³⁺ or Cu²⁺ (1 μM) and a H₂O₂ trapping agent, tris(2-carboxyethyl)phosphine hydrochloride (TCEP, Pierce, 50 μM), are co-incubated in PBS buffer (300 mL, pH 7.4), for 1 hour at 37° C. in the presence or absence of the inventive bifunctional molecule to be tested (1-5 μM). Under identical conditions, catalase (Aldrich-Sigma, 100 U/mL) is substituted for the peptide, to serve as a control signal representing no H₂O₂. Following incubation, the unreacted TCEP is detected by 5,5-dithio-bis(2-nitrobenzoic acid) (DTNB, Aldrich-Sigma) which generates 2 moles of the colored product, 2-(nitro-5-thiobenzoate) (NTB). The reactions are, as follows:
TCEP+H₂O₂→TCEP=O+H₂O
then the remaining TCEP is reacted with DTNB:
TCEP+DTNB+H₂O→TCEP=O+2 NTB

The amount of H₂O₂ produced is quantified based on the following formula: H₂O₂ (μM)=(ΔA×10⁶)/(2×l×ε), where ΔA* is the absolute absorbance difference between a sample and catalase-only control at 412 nm; l=0.875 cm, the equivalent vertical pathlength obtained from the plate-reader manufacturer's specifications; and ε is the molecular absorbance for NTB (14,150 M⁻¹cm⁻¹ at 412 nm). TCEP is a strong reducing agent that can artifactually react with polypeptides that contain disulfide bonds. However, this reaction cannot take place with Aβ, since Aβ does not possess such chemical bonds.

Assay for the Detection of O₂.⁻. The production of O₂.⁻ can be estimated by measuring (using a 96-well plate reader) the absorption of the Aβ peptide (Aβ₁₋₄₂ or Aβ₁₋₄₀, 10 μM, 300 μL per well) after incubation for one hour in PBS (pH 7.4) at 37° C., in the presence or absence of the inventive bifunctional molecule to be tested (1-5 μM). The corresponding blank is the signal from PBS alone. An absolute baseline for the signal generated by the peptide is not achievable in this assay since the absorption peak for tyrosine (residue 10 of Aβ) is close (254 nm) to the absorption peak for O₂.⁻. However, attenuation of the absorbance by co-incubation with superoxide dismutase (100 U/mL) helps ascertain that the majority of the absorbance signal is due to the presence of O₂.⁻.

Thiobarbituric Acid Reaction Substance (TBARS) Assay for the Detection of .OH. The Thiobarbituric Acid-Reactive Substance (TBARS) assay for incubation mixtures with Fe^{3+ or Cu}²⁺ can be performed in a 96-well microtiter format modified from established protocols (J. M. Gutteridge et al., Biochim. Biophys. Acta, 1983, 759: 38-41). The amyloid-β peptide (Aβ₁₋₄₂ or Aβ1-40; 10 μM) or Vitamin C (100 μM), is incubated with Fe³⁺ or Cu²⁺ (1 μM) and deoxyribose (7.5 mM, Aldrich-Sigma) in PBS (pH 7.4) in the presence or absence of the inventive bifunctional molecule to be tested. Following incubation (37° C., 1 hour), glacial acetic acid and 2-thiobarburic acid (1%, w/v in 0.05 M NaOH, Aldrich-Sigma) are added and heated (100° C., 10 min). The final mixtures are placed on ice for 1-3 minutes before absorbances at 532 nm are measured. The net absorbance change for each sample is obtained by deducting the absorbance from a control sample consisting of identical chemical components except for the Vitamin C or Aβ peptides.

Resolubilization of Metal-Induced Aβ Aggregates

To assess the ability of an inventive bifunctional molecule to cause resolubilization of metal-induced Aβ aggregates, Aβ (Aβ₁₋₄₀ or Aβ₁₋₄₂; 10 ng/well in PBS) aggregation is first induced by addition of ZnCl₂ (25 μM), or CuCl₂ (5 μM). Aggregates are then transferred to a 0.22 μm nylon membrane by filtration. The aggregates are then washed (200 μL/well) with PBS alone, or PBS containing 2 μM of the inventive bifunctional molecule to be tested, or PBS containing 2 μM of Clioquinol, used as control. The membrane is fixed, probed with the anti-Aβ monoclonal antibody 6E10, and developed for exposure to ECL-film. Relative signal strength is determined by densitometric analysis of the ECL-film, calibrated against known amounts of the peptide. Values are expressed as a percentage of Aβ signal remaining on the filter after washing with PBS alone.

### Example 5

### Cell-Based Assays to Test Bifunctional Molecules

Neurotoxicity of Bifunctional Molecules

(a) E17 Rat Cortical Primary Neurons

A primary neuronal culture was used to test the neurotoxicity of the parent bifunctional molecule, XH1. E17 rat cortical primary neurons were obtained from pathogen-free female Sprague-Dawley rats (purchased from Taconic Farms, Mass.) after 17 days of gestation, as described by G. J. Brewer and C. W. Cotman (in Brain Res. 1989, 494: 65-74). The protocol used allowed long-term culture of neurons at low density under precisely defined culture conditions. This protocol provided up to 90% neuronal culture. It was desirable that a small population of glial cells be co-cultured with the rat cortical primary neurons as these cells support neuronal survival. Cytosine arabinoside (1 μM) was used to control the growth of glial cells in the culture preparation. The neuronal population was checked regularly using neuron-specific enolase (and/or astroglia-specific S100β) immunohistochemistry.

E17 rat cortical primary neurons were grown under 95% O₂, 5% CO₂, 85% humidity conditions, at 37° C. for 4 days in serum-free Neurobasal™ medium with B-27 supplement (Life Technologies, Inc.), 20 μM L-glutamate, 100 units/mL penicillin, 0.1 g/mL streptomycin, and 2 mM L-glutamine. On the 5^{th} day (treatment day), the medium was replaced with serum-free Neurobasal™ medium plus L-glutamine without B-27 supplement. The cells were then incubated in the presence of XH1 (0, 1 or 10 μM) and cell viability was assayed 48 hours after treatment with XH1. LDH release assays and MTT assays were performed using kits commercially available from Roche (Nutley, N.J.) to assess necrotic cell death, and to determine cellular metabolic activity, respectively.

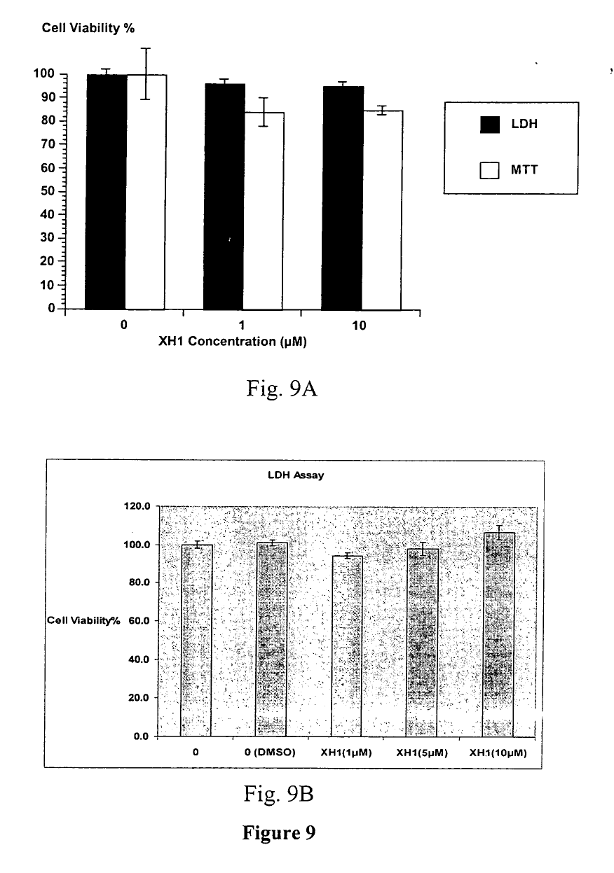

Results of these assays are reported in FIG. 9A as mean values calculated using data obtained in three sets of experiments. The results show that XH1 does not induce any significant cell death at low macromolecular concentrations in E17 rat cortical primary neurons.

(b) Human SH-SY5Y Neuroblastoma Cells

The neurotoxicity of XH1 was also tested on human SH-SY5Y neuroblastoma cells. The SH-SY5Y cell line is commonly used to study neuritogenesis, differentiation, and tumorigenesis (D. Vu et al., Brain Res. Mol. Brain Res. 2003, 115: 93-103). SH-SY5Y cells were obtained from American Type Culture Collection and grown in Dulbecco's modified Eagle's medium supplemented with 10% fetal bovine serum (Gibco-BRL) and antibiotics.

Human SH-SY5Y neuroblastoma cells were incubated in the presence of XH1 (0, 1, 5 or 10 μM) (95% O₂, 5% CO₂, 85% humidity, 37° C.) and cell viability was assessed 48 hours after treatment using the LDH release assay as described above. The results of these experiments, which are reported in FIG. 9B, show that XH1 does not cause any significant cell death at low macromolecular concentrations in human SH-SY5Y neuroblastoma cells.

Effects of Bifunctional Molecules on Metal-Regulated APP Protein Expression

Several facts suggest a direct connection between increased levels of the amyloid protein precursor (APP) and the development of Alzheimer's disease. APP has been associated with AD because it is processed through proteolytic cleavage into the β-peptide that accumulates in amyloid plaques, and because APP gene mutations can cause early onset AD. However, even in the presence of Familial AD mutations, over-expression of APP in transgenic mice was found to be necessary for sufficient Aβ peptide production to lead to the development of amyloid filament deposits and an Alzheimer's-like pathology. There are now several reports supporting an important role for translational regulatory mechanisms to control APP synthesis and probably Aβ peptide secretion in biologically relevant conditions (van Leeuwen et al., Science, 1998, 279: 242-247).

Different experiments can be performed to assess the ability of XH1 and its analogues to decrease or inhibit APP synthesis and consequential Aβ production.

(a) Determination of APP Protein Synthesis

In SH-SY5Y human neuroblastoma cells, the capacity of XH1 to suppress APP synthesis was measured by SDS-PAGE. Control proteins (β-tubulin and/or two amyloid-precursor-like proteins, APLP1 and APLP2) were used to ensure target suppression specificity of the inventive bifunctional molecule.

SDS-PAGE. SH-SY5Y human neutoblastoma cells were harvested and washed with cold PBS three times and then lysed with M-PER™ mammalian protein extraction reagent (Pierce) mixed with protease inhibitor cocktails (Roche). The cell lysate was vortexed for 15 minutes at 13,000 rpm in a cold room, and the supernatants were BCA assayed for total protein concentrations. Protein blotting was performed on pre-casted NuPAGE™ 4-12% Bis-Tris Gel (Invitrogen) with equal protein loading (15 μg/well). It was run at 200 V for 45 minutes and transferred at 75 mA/gel for 95 minutes. The blots were probed by primary and secondary antibodies according to manufacturer's instructions and finally washed for 2-3 hours at 15 min. intervals. The blots were then developed using a chemiluminescent kit (Pierce).

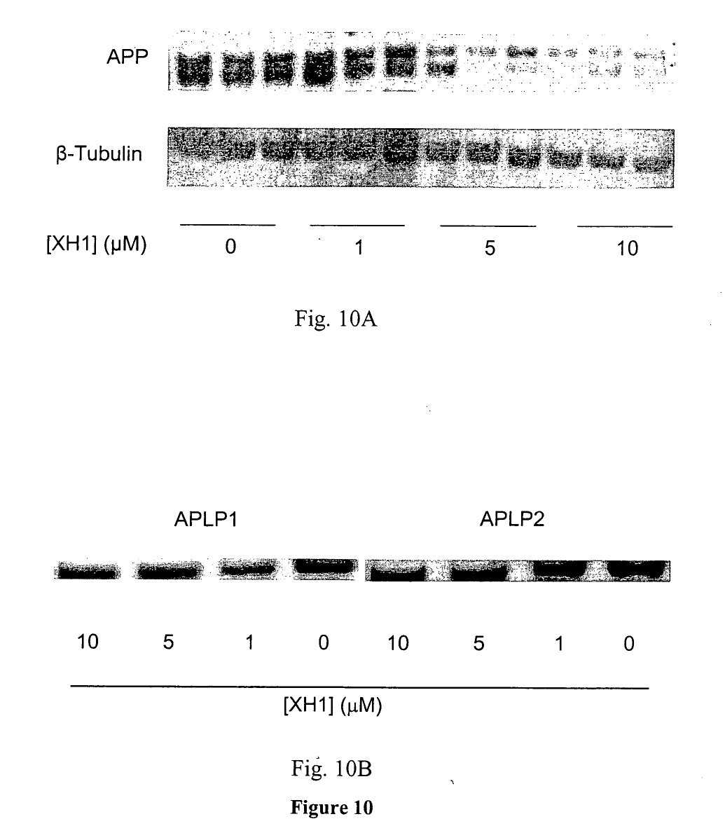

The results of these experiments are reported in FIG. 10. As shown in FIG. 10A, an increase in XH1 concentration led to a decrease in APP synthesis while under the same conditions, no effects were seen on β-tubulin expression. Similarly, as shown in FIG. 10B, the presence of XH1 (up to a concentration of 10 μM) did not affect APLP1 or APLP2 synthesis. These data demonstrate the target suppression specificity of XH1.

Metabolic Labeling of Neuroblastoma Cells. Intracellular APP protein synthesis can be determined in primary neuroblastoma after plating cells in equal numbers into 8 microtiter wells prior to each treatment (1×10⁵ cells per well in 96 well dishes). Cells are treating for 48 hours in the presence of XH1, DPTA, or left untreated. Cells from random wells are counted in order to ensure a consistent presence of 1×10⁵ cells per well at the beginning of each experiment. Neuroblastoma cells are preincubated for 15 minutes in methionine-free medium and pulse-labeled with 300 μCi/mL [³⁵S]-methionine for 30 minutes in methionine-free medium (RPMI 1640; GIBCO). Each microtiter plate is washed twice in cold PBS at 4° C. before lysis of neuroblastoma cells with 25 mL STEN buffer and a sterile glass rod (STEN buffer was 0.2% NP-40, 2 mM EDTA, 50 mM Tris, pH 7.6). The addition of 20 mM PMSF, 5 mg/mL leupeptin to the lysis buffer prevents proteolysis. The buffers from each well are pooled into a total volume of 300 μL. One half of each pooled lysate is immunoprecipitated with antiserum raised against the carboxyl terminus of APP (1:500 dilution of C-8 antibody raised against amino acid residues 676-695 of APP-695). The remaining portion of each lysate is immunoprecipitated with human ferritin antiserum (1:500 dilution, Boehringer).

In all labeling experiments, the immunoprecipitated protein is collected through the binding of antibody-labeled antigen complexes to Protein A Sepharose™ beads. Immuno-precipitated samples are applied to 10-20% Tris-Tricine gels (Novex) and the samples are electrophoresed in Tris-Tricine buffer according to the manufacturer's instructions. The gels are fixed with 25% methanol, 7% (v/v) methanol for 1 hour, treated with fluorographic reagent (Amplify, Amersham) for 30 minutes, dried, and exposed to X-omat Kodak film overnight at −80° C.

(b) Determination of APP mRNA Levels

For comparison, the ability of XH1 to affect APP processing can be assessed by measuring mRNA levels of APP by real-time RT-PCR.

(c) Determination of Intracellular and Secreted Aβ₁₋₄₀ and Aβ₁₋₄₂ Concentrations

Both intracellular and secreted concentrations of Aβ₁₋₄₀ and Aβ₁₋₄₂ after treatment of SH-SY5Y cells with XH1 can be determined using a commercial ELISA kit for Aβ_{40/42} levels (BioSource International).

(d) Screening Assay

The 5′-untranslated region (5′-UTR) of the mRNA coding for APP was demonstrated to contain iron-responsive elements (J. T. Rogers et al., J. Biol. Chem. 2002, 47: 45518-45528), i.e., RNA stem-loops that control cellular iron homeostasis by regulating ferritin translation and transferritin receptor mRNA stability. Iron levels have been shown to regulate APP mRNA translation in astrocytic cells (J. T. Rogers et al., J. Biol. Chem. 1999, 274: 6421-6431), while intracellular iron levels have been found to regulate APP synthesis in neuroblastoma cells (J. T. Rogers et al., J. Biol. Chem. 2002, 47: 45518-45528). Furthermore, the presence of an iron chelator such as desferrioxiame was reported to induce a down-regulation of APP 5′-UTR conferred translation, which was observed to be reversed by iron influx.

J. T. Rogers and coworkers (J. Mol. Neurosci. 2002, 19: 77-82) have developed a transfection based assay to screen potential drugs for their ability to inhibit APP expression by interacting with the 5′-UTR of the mRNA coding for APP. They have used this assay to screen different classes of drugs, including known blockers of receptor ligand interactions, bacterial antibiotics, drugs involved in lipid metabolism, and metal chelators.

In collaboration with Dr. Rogers, assays similar to that earlier reported will be used to test the ability of XH1 and its analogues to decrease the APP expression and consequential Aβ production through interaction with the mRNA 5′-UTR.

Constructs Preparation. pSV2(APP)Luciferase and pSV2(APP)GFP constructs will be made by fusing 5′-UTR sequences of APP gene with downstream reporter genes (Luciferase and green fluorescence protein, GFP), respectively, as described in J. T. Rogers et al., J. Mol. Neurosci. 2002, 19: 77-82.

Transfections. Human SH-SY5Y neuroblastoma cells will be transfected with 10 μg of DNA from the pGL-3, pGAL and pGALA constructs and co-transfected with 5 μg of DNA from a construct that expresses green fluorescence protein (GFP). Luciferase and GFP reporter genes are expressed from an SV40 promoter. Transfections will be performed in the presence of LipofectAMINE-2000 according to the manufacturer's instructions (Invitrogen). Typically, neuroblastoma cells are grown in flasks (100 mm²) for each treatment. Each flask is transfected (12 h) and subsequently passaged equally into 96-well plates for exposure to XH1 (or another bifunctional molecule to be tested) and desferrioxamine as control for 48 hours for each treatment.

After treatment for 48 hours, cell viability will be established by a microscopic examination of each well. Cell viability will be confirmed by relative expression of GFP in each 96-well by reading at 480/509-nm wavelength (GFP) using an automated Wallac 1420 multilabel counter. After obtaining a GFP readout the cells in each 96-well plate will be lysed in 50 μL of reporter lysis buffer (Promega, Madison, Wis.) followed by luciferase assays using the Wallac 1420 counter.

To rule out the possibility that the inventive bifunctional molecules exhibit non-specific down-regulatory effects on overall translation, it will be necessary to also use constructs that express antisense versions of the APP 5′UTR sequences in front of reporters (i.e., APP, CAT, Luciferasse, and GFP). Transfection with these constructs will ensure that the screened compounds are targeted with high selectivity to the correct secondary structure of APP-mRNA. The gold standard for specificity will be to check whether APLP1 and APLP2 synthesis and 5′-UTR-driven gene expression remains unchanged while APP gene expression, synthesis, and AD levels are repressed.

### Example 6

### Ex Vivo Dissolution of Aβ Amyloid by Inventive Bifunctional Molecules

The assay described below can be used to assess the ability of inventive bifunctional molecules to extract Aβ deposits from human brain tissue.

Preparation of Samples. Post-mortem tissues, stored at −80° C., are obtained together with accompanying histopathological and clinical data, with half of the samples from patients with Alzheimer's disease, the other half from age-matched healthy patients. Alzheimer's disease is assessed according to Consortium to Establish a Register for Alzheimer's Disease (CERAD) criteria (S. S. Mirra et al., Neurology, 1997, 49: S14-16), with particular attention paid to the presence of neuritic plaques and neurofibrillary tangles.

Extraction of Aβ Amyloid from Post-mortem Brain Tissue. Identical regions of frontal cortex (0.5 g) are homogenized using a DIAX 900 homogenizer (Heidolph & Co, Kelheim, Germany) for three 30-s periods at full speed, with a 30-s rest between strokes, in 3 mL of ice-cold PBS (pH 7.4), containing a mixture of protease inhibitors (BioRad, Hercules, Calif.) with the exception of EDTA, or in the presence of 0.1 to 2 mM of either an inventive bifunctional molecule or Clioquinol, used as control. To obtain the PBS-extractable fraction, the homogenates are centrifuged at 100,000 xg for 30 minutes (Beckman J180, Beckman instruments, Fullerton, Calif.), and the supernatant is collected, divided into 1-mL aliquots and stored on ice or immediately frozen at −70° C. Protein within a 1-mL supernatant sample is precipitated using 1:5 ice-cold 10% trichloroacetic acid, and pelleted by centrifugation at 10,000×g for 20 minutes. The pellet is prepared for polyacrylamide gel electrophoresis by boiling for 10 minutes in Tris-Tricine SDS-sample buffer containing 8% SDS, 10% mercaptoethanol, and 8 M urea. Total Aβ in the cortical samples is obtained by homogenizing in 1 mL of PBS and boiling in sample buffer as above.

Polyacrylamide Gel Electrophoresis and Western Blotting. Tris-Tricine polyacrylamide gel electrophoresis is performed by loading samples onto 10-well, 10-20% gradient gels (Novex, San Diego, Calif.), followed by Western transfer onto a 0.2-mm nitrocellulose membrane (BioRad, Hercules, Calif.). The Aβ peptide is detected using monoclonal antibodies WO2 (which detects Aβ₁₋₄₀ and Aβ₁₋₄₂ at an epitope between 5 and 8), G210 (which is specific for Aβ species that terminate at carboxyl residue 40) or G211 (which is specific for Aβ species that terminate at residue 42) (N. Ida et al., J. Biol. Chem. 1996, 271: 22908-22914), in conjunction with horseradish peroxidase-conjugated rabbit anti-mouse IgG (Dako, Denmark) and visualized using chemiluminescence (ECL, Amersham Biosciences Corp., Piscataway, N.J.). Each gel includes two or more lanes containing known quantities of synthetic Aβ which serve as internal reference standards.

Blot Scanning and Transmission Densitometry Assay for Aβ. Blot images are scanned using a Relisys scanner with transparency adapter (Teco Information Systems, Taiwan), and densitometry is performed using Image 1.6 software (NIH, Bethesda, Md.) modified for PC (by Scion Corporation, Frederick, Md.), calibrated using a step diffusion chart. For quantification of Aβ in brain extracts, the internal reference standards of synthetic Aβ are utilized to produce standard curves from which values are interpolated.

### Example 7

### Properties of Inventive MRI Contrast Imaging Agents

MRI Signals from Spherical Phantoms

Different solution mixtures (containing a contrast imaging agent, Gd-XH1 or Gd-DTPA, in the presence or absence of Aβ₁₋₄₀, Aβ₁₋₄₂ or HSA in PBS, pH 7.4) were injected into 4.5-mL hollow spheres (made of polypropylene) and subjected to a 3-T magnetic scanner (Simens). Spin-Echo scanning mode was used and T1 signals were measured for each solution using 12 TR values (TE=5 ms), with R1=1/T1, and R1(obs)=R1(O)+R1 (Gd-XH1-Aβ₁₋₄₀ or -Aβ₁₋₄₂).

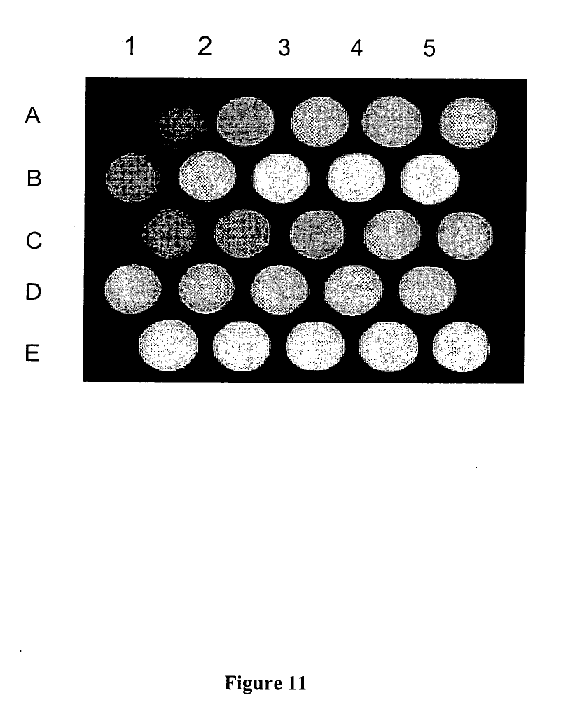

The results of these experiments, which are reported in FIG. 11, show the effects of Gd-XH1 on longitudinal (T1) magnetic relaxation rates. As the concentration of Gd-XH1 increases, T1 decreases and the signal gets brighter. No effects were observed for the combination of Gd-DTPA (small hydrophilic molecule used as control) with HSA or Aβ.

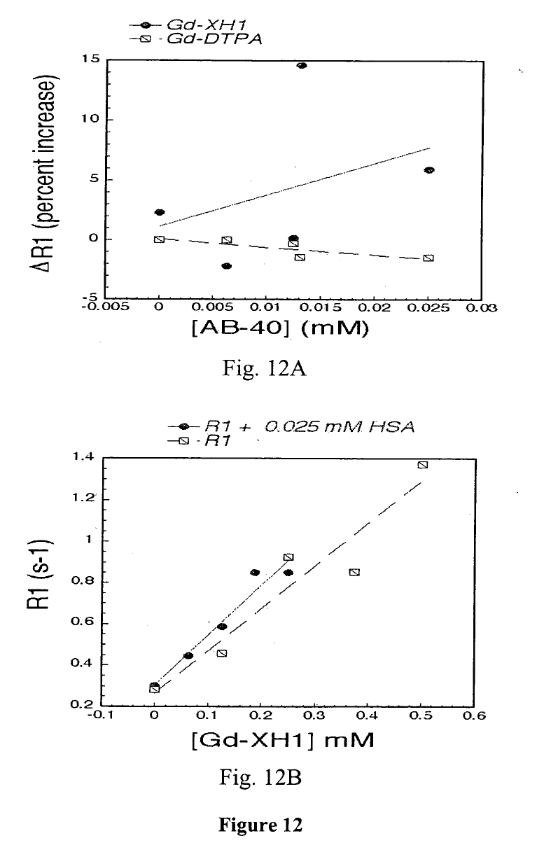

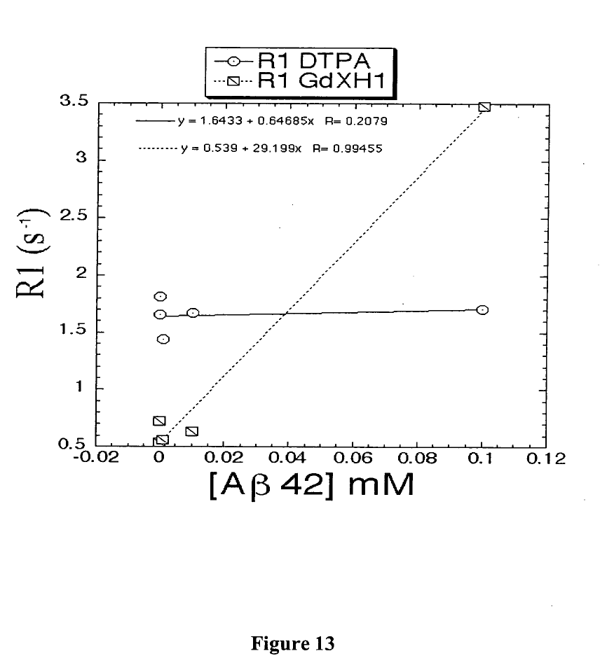

The results of these experiments are also reported in FIG. 12 and FIG. 13. They indicate that Gd-XH1 specifically interacts with Aβ₁₋₄₀ peptide while it only interacts slightly with HSA (FIG. 12). In addition, as shown in FIG. 13, there is an increase in the relaxation rate of Gd-XH1 when bound to Aβ₁₋₄₂, which is rich in β-sheets, compared to when bound to native Aβ₁₋₄₀, which exhibits a lower β-sheet content.

MRI Signals from AD Mouse and Human Brain Tissue Extracts

Both AD mouse (PS1(M146V)xAPPTg2576) and human brain tissue extracts were prepared by lysing the tissues with T-PER™ tissue protein extraction reagent (Pierce) mixed with protease inhibitor cocktails (Roche). Then the different solution mixtures containing 0.25 mM Gd-XH1 and 10 μg/mL (total protein) extracts were injected into 4.5-mL hollow spheres and subjected to the same experimental protocol described above.

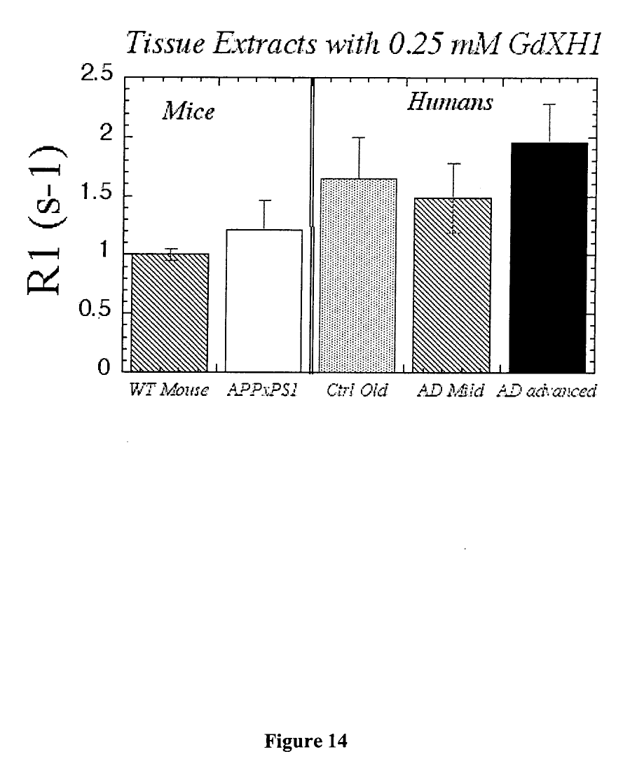

The results of these experiments are reported in FIG. 14. They show that MRI signals are enhanced in AD mouse and human brain tissue extracts when mixed with Gd-XH1.

### Example 8

### MRI Detection of Aβ Amyloid Deposits in an Animal Model of AD

A method for detecting the presence of amyloid deposits in an animal model of Alzheimer's disease is described herein. The method was based on the use of Gd-XH1. As shown above, Gd-XH1 specifically interacts with Aβ₁₋₄₀ and Aβ₁₋₄₂. Furthermore, MRI signals from AD mouse and human brain tissue extracts were found to be enahnced when the extracts were mixed with Gd-XH1.

The animal model used in this series of experiments was the transgenic Tg2576 mice strain, which over-expresses the human amyloid precursor protein (APP) with a familial AD gene mutation, and exhibits neuropathology characteristic of AD such as memory deficits and age-related formation of amyloid deposits in specific regions of the brain. The imaging was carried out using a small 9.4 T MRI system (400 MHz; Magnex Scientific, Kidlington, UK) at the MGH/MIT/HMS Athinoula A. Martinos Center for Functional and Structural Biomedical Imaging (Department of Radiology, Massachusetts General Hospital, Boston, Mass.).

To first assess the toxicity of Gd-XH1 in vivo, PS1(M146V)xAPPTg2576 mice (about 6 months old; 5 mice per group) were daily injected intraperitoneally with a dose of 30 mg per kg of body weight of Gd-XH1 for 4 weeks. Controls include injection of the same dose of Gd-DTPA, and no treatment. No acute toxicity of Gd-XH1 was observed.

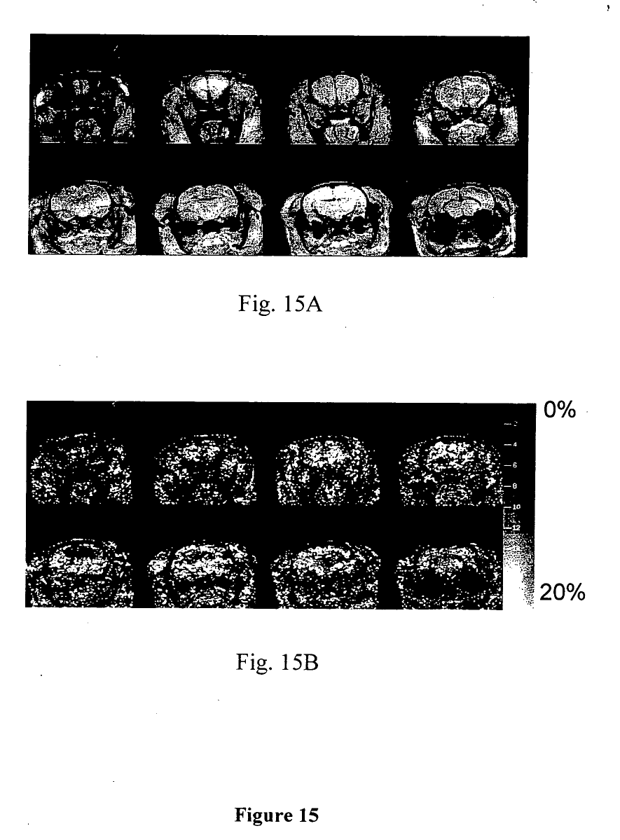

A first series of test experiments was carried out to assess the MRI contrast imaging ability of Gd-XH1. Gd-XH1 was mixed with methyl cellulose and prepared as a suspension. One male Sprague-Dawley rat (8 months old) was injected intraperitoneally with a single dose of 10 mg per kg of body weight of this suspension. One hour after injection, the animal was imaged with a 4.7 T MRI instrument (GE) using a sequential scanning mode. Both anatomic and S/N ratiometric MRI images were recorded. Some of the images obtained are shown in FIG. 15. One hour after i.p. injection, a 8% increase of the signal to noise ratio was observed. The increase in T1-weighted signal intensity appear to be widespread, indicating that Gd-XH1 is both BBB and skeletal muscle permeable.

Further experiments will be carried out using APP transgenic mice instead of rats. More specifically, a group of 5 to 10 APP transgenic Tg2576 mice will be injected intraperitoneally with a single dose of 0.1 mmol per kg of body weight of an inventive MRI contrast imaging agent, for example Gd-XH1, in a mixture of DMSO and PBS (60:40; v:v). For comparison, another group of 5 to 10 PS1(M146V)xAPPTg2576 mice will be injected intraperitoneally with a single dose of 0.1 mmol per kg of body weight of a control contrast imaging agent in PBS. The control imaging agent will be such that it comprises the same metal-chelating moiety complexed to the same paramagnetic metal ion than the inventive contrast imaging agent but, contrary to the inventive contrast imaging agent, does not comprise any amyloid-binding moieties. For example, in the case of the contrast imaging agent Gd-XH1, Gd-DTPA will be used as control. After injection, both groups of animals will be imaged by MRI. The effects of the inventive MRI contrast imaging agent on enhancement of cerebral Aβ amyloid images in the transgenic mice will be assessed and confirmed by Aβ immuno-staining after sacrifice of the animals.

### Example 9

### Synthesis of a Family of Bifunctional Molecules Comprising an α-Lipoic Acid Moiety

A second family of novel bifunctional molecules has been designed and is being developed. The new bifunctional molecules comprise one metal-chelating moiety directly, covalently linked to one amyloid-binding moiety. The metal-chelating moiety, which is common to all the bifunctional molecules of the family, is α-lipoic acid, which is also known to have powerful anti-oxidant properties. As shown in FIG. 6A, the amyloid-binding moiety of the parent molecule of the family is the same benzothioflavin derivative than the one used in the first family of bifunctional molecules (Example 1).

Other members of the family are analogues of the parent bifunctional molecule, in which the aromatic ring of the benzothiazole moiety is substituted with one or more than one functional group including, for example, 4-dimethylamino; 4-amino; 4-chloro; 4-chloro-5-ethyl; 4-acetyl; 5-carboxyl; 5-sulfonoxyl; 5-bromo; 4-, 5- or 6-methyl; 5-trifluoromethyl; 4-ethoxyl; 4-, 5- or 6-methylsulfonyl; and 4-, 5- or 6-hydroxyl (see FIG. 6B).

The synthesis of the parent bifunctional molecule of the family (compound XH2) is described herein. The preparation of XH2 involves the formation of amide bonds between the metal-chelating moiety and the amyloid-binding moieties. The reaction was carried out according to a procedure adapted from previously published synthetic methods (W. E. Klunk et al., Life Sci. 2001, 69: 1471-1484; D. Shi et al., J. Med. Chem. 1996, 39: 3375-3384; and M. S. Konings et al., Inorg. Chem. 1990, 29: 1488-1491).

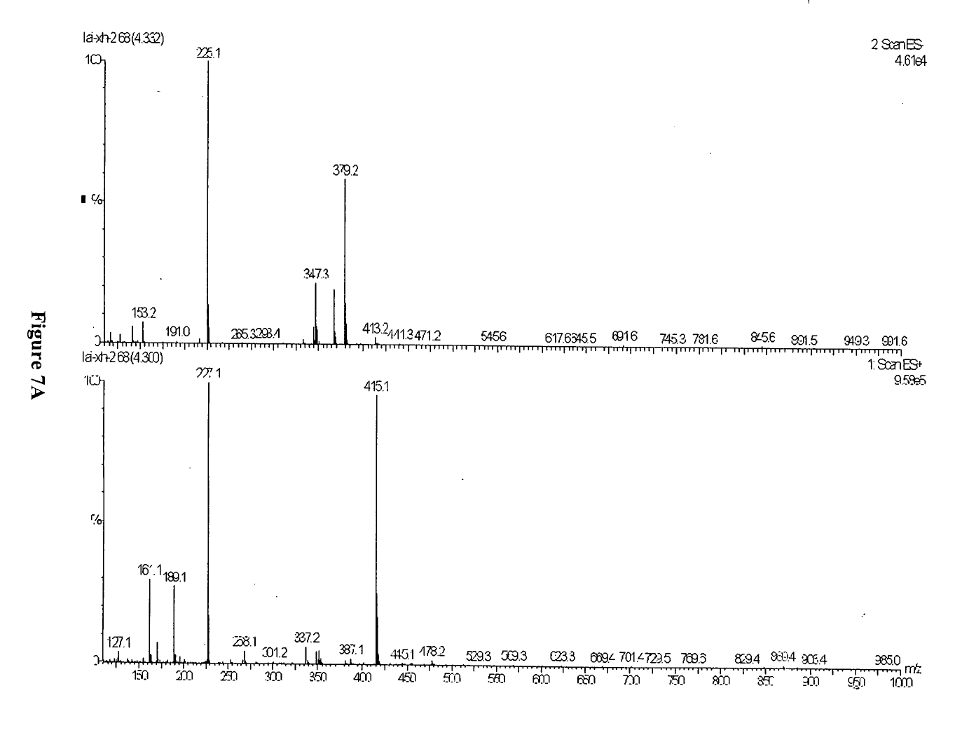

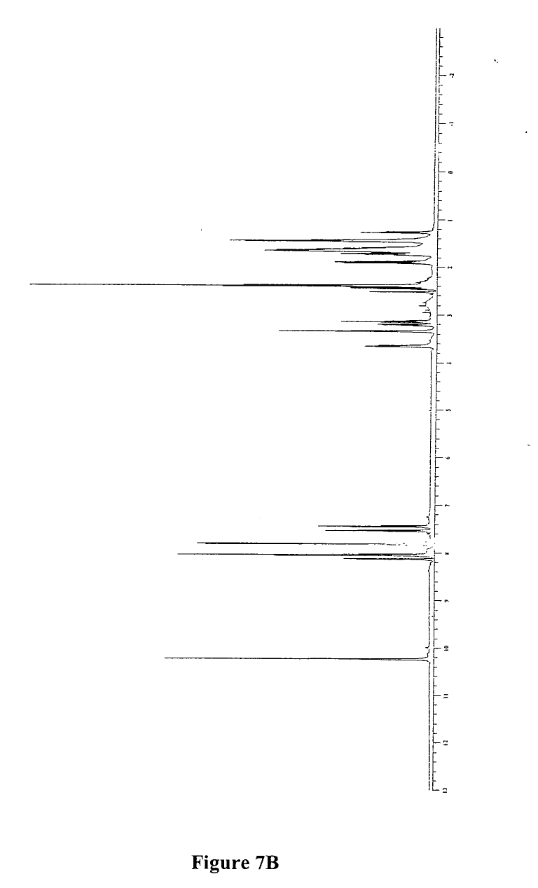

To a stirred solution of 1 mg lipoic acid (Aldrich Chemical) in methylene chloride at room temperature was added two drops of DMF. To this solution was added, dropwise, 0.5 mL oxalyl chloride. The surface of the reaction showed generation of a small amount of gas. When the bubbling ceased, the mixture was treated with a solution of 1.2 mg of 4-benzothiazol-2-yl-phenylamine and 1 mL of N,N-diisopropylethylamine in 15 mL methylene chloride. The dark green mixture was stirred at room temperature for one hour. The mixture was washed with water, dried over sodium sulfate, filtered and evaporated to afford a solid. This was triturated with ethyl acetate and dried under vacuum to afford XH2 (90%), the structure of which was confirmed by ¹H-NMR (see FIG. 7).

What is claimed is:

1. A bifunctional molecule comprising at least one metal-chelating moiety associated with at least one amyloid-binding moiety, wherein the amyloid-binding moiety comprises a derivatized benzothiazole, and the metal-chelating moiety comprises DTPA.

2. The bifunctional molecule of claim 1, wherein the bifunctional molecule has the following chemical structure: 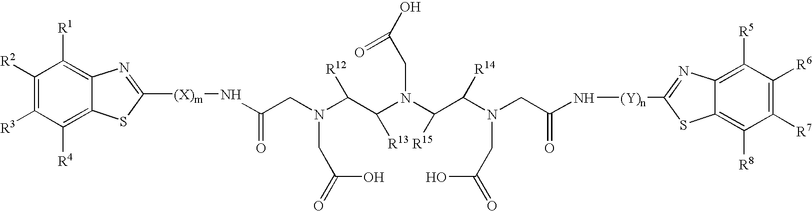
wherein
R¹, R², R³, R⁴, R⁵, R⁶, R⁷ and R⁸ are each independently selected from the group consisting of hydrogen, halogen, cyano, (C₁₋₆)alkyl, (C₃₋₇)cycloalkyl, trifluoromethyl, amino, alkylamino, dialkylamino, hydroxy, nitro, COOH, COOR⁹, CONHR¹⁰, CONR¹¹, hydroxyethyl and oligomers and alkyl ethers thereof, aryloxy, substituted aryloxy, heteroaryloxy, substituted heteroaryloxy, heterocycloalkyloxy, substituted heterocycloalkyloxy, thioalkyl, carbonyl, thiocarbonyl, benzyl, carboxamido, sulfonamido, aryl, substituted aryl, heteroaryl, substituted heteroaryl, heterocycloalkyl and substituted heterocycloalkyl;
R⁹, R¹⁰ and R¹¹ are each independently (C₁₋₆)alkyl:
R¹², and R¹³ are each independently selected from the group consisting of hydrogen, halogen, cyano, (C₁₋₆)alkyl, (C₃₋₇)cycloalkyl, trifluoromethyl, amino, alkylamino, dialkylamino, hydroxy, nitro, COOH, COOR⁹, CONHR¹⁰, CONR¹¹ (where R⁹, R¹⁰ and R¹¹ are as previously defined), hydroxyethyl and oligomers and alkyl ethers thereof, aryloxy, substituted aryloxy, heteroaryloxy, substituted heteroaryloxy, heterocycloalkyloxy, substituted heterocycloalkyloxy, thioalkyl, carbonyl, thiocarbonyl, benzyl, carboxamido, sulfonamido, aryl, substituted aryl, heteroaryl, substituted heteroaryl, heterocycloalkyl and substituted heterocycloalkyl, or when taken together form a 5, 6 or 7 member carbocyclic ring or a heterocyclic ring containing oxygen, sulfur or nitrogen;
R¹⁴, and R¹⁵ are each independently selected from the group consisting of hydrogen, halogen, cyano, (C₁₋₆)alkyl, (C₃₋₇)cycloalkyl, trifluoromethyl, amino, alkylamino, dialkylamino, hydroxy, nitro, COOH, COOR⁹, CONHR¹¹, CONR¹¹ (where R⁹, R¹⁰ and R¹¹ are as previously defined), hydroxyethyl and oligomers and alkyl ethers thereof, aryloxy, substituted aryloxy, heteroaryloxy, substituted heteroaryloxy, heterocycloalkyloxy, substituted heterocycloalkyloxy, thioalkyl, carbonyl, thiocarbonyl, benzyl, carboxamido, sulfonamido, aryl, substituted aryl, heteroaryl, substituted heteroaryl, heterocycloalkyl and substituted heterocycloalkyl, or when taken together form a 5, 6 or 7 member carbocyclic ring or a heterocyclic ring containing oxygen, sulfur or nitrogen;
X and Y are each independently divalent linking moieties selected from the group consisting of (C₁₋₆)alkyl, (C₃₋₇)cycloalkyl, heterocyclic, substituted heterocyclic, heterocycloalkyl, substituted heterocycloalkyl aryl and substituted aryl; and
m and n are each independently 0 or 1,
with the proviso that the bifunctional molecule does not have the following structure:

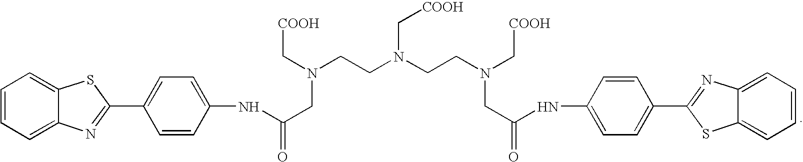

3. The bifunctional molecule of claim 2, wherein X and Y are independently phenyl or substituted phenyl, and wherein the phenyl or substituted phenyl carries the group: 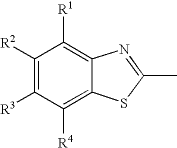
at the ortho, meta or para position.

4. A bifunctional molecule comprising at least one metal-chelating moiety associated with at least one amyloid-binding moiety, wherein the amyloid-binding moiety comprises a derivatized benzothiazole, and the metal-chelating moiety comprises an α-lipoic acid derivative.

5. The bifunctional molecule of claim 4, wherein the bifunctional molecule has the following chemical structure: 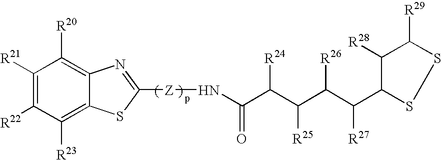
wherein
R²⁰, R²¹, R²², R²³, R²⁴, R²⁵, R²⁶, R²⁷, R²⁸ and R²⁹ are independently selected from the group consisting of hydrogen, halogen, cyano, (C₁₋₆)alkyl, (C₃₋₇)cycloalkyl, trifluoromethyl, amino, alkylamino, dialkylamino, hydroxy, nitro, COOH, COOR⁹, CONHR¹⁰, CONR¹¹ (where R⁹, R¹⁰ and R¹¹ are as previously defined), hydroxyethyl and oligomers and alkyl ethers thereof, aryloxy, substituted aryloxy, heteroaryloxy, substituted heteroaryloxy, heterocycloalkyloxy, substituted heterocycloalkyloxy, thioalkyl, carbonyl, thiocarbonyl, benzyl, carboxamido, sulfonamido, aryl, substituted aryl, heteroaryl, substituted heteroaryl, heterocycloalkyl and substituted heterocycloalkyl;
Z is a divalent linking moiety selected from the group consisting of (C₁₋₆)alkyl, (C₃₋₇)cycloalkyl, trifluoromethyl, amino, alkylamino, dialkylamino, hydroxy, nitro, COOH, COOR⁹, CONHR¹⁰, CONR¹¹ (where R⁹, R¹⁰ and R¹¹ are as previously defined), hydroxyethyl and oligomers and alkyl ethers thereof, aryloxy, substituted aryloxy, heteroaryloxy, substituted heteroaryloxy, heterocycloalkyloxy, substituted heterocycloalkyloxy, thioalkyl, carbonyl, thiocarbonyl, benzyl, carboxamido, sulfonamido, aryl, substituted aryl, heteroaryl, substituted heteroaryl, heterocycloalkyl and substituted heterocycloalkyl; and
p is 0 or 1;
with the proviso that the bifunctional molecule does not have the following structure:

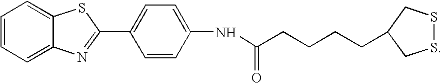

6. The bifunctional molecule of claim 5, wherein Z is phenyl or substituted phenyl, and wherein the phenyl or substituted phenyl carries the group: 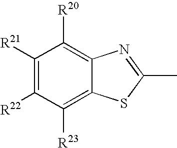
at the ortho, meta or para position.

7. A contrast imaging agent comprising a bifunctional molecule complexed to at least one metal entity, wherein the bifunctional molecule comprises at least one metal-chelating moiety associated with at least one amyloid-binding moiety, wherein the amyloid-binding moiety comprises a derivatized benzothiazole, and the metal-chelating moiety comprises DTPA.

8. The contrast imaging agent of claim 7, wherein the bifunctional molecule has the following structure: 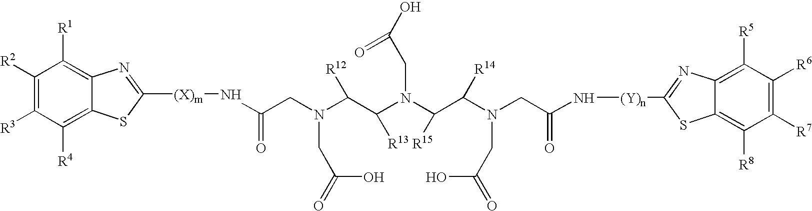
wherein
R¹, R², R³, R⁴, R⁵, R⁶, R⁷ and R⁸ are each independently selected from the group consisting of hydrogen, halogen, cyano, (C₁₋₆)alkyl, (C₃₋₇)cycloalkyl, trifluoromethyl, amino, alkylamino, dialkylamino, hydroxy, nitro, COOH, COOR⁹, CONHR¹⁰, CONR¹¹, hydroxyethyl and oligomers and alkyl ethers thereof, aryloxy, substituted aryloxy, heteroaryloxy, substituted heteroaryloxy, heterocycloalkyloxy, substituted heterocycloalkyloxy, thioalkyl, carbonyl, thiocarbonyl, benzyl, carboxamido, sulfonamido, aryl, substituted aryl, heteroaryl, substituted heteroaryl, heterocycloalkyl and substituted heterocycloalkyl;
R⁹, R¹⁰ and R¹¹ are each independently (C₁₋₆)alkyl:
R¹², and R¹³ are each independently selected from the group consisting of hydrogen, halogen, cyano, (C₁₋₆)alkyl, (C₃₋₇)cycloalkyl, trifluoromethyl, amino, alkylamino, dialkylamino, hydroxy, nitro, COOH, COOR⁹, CONHR¹⁰, CONR¹¹ (where R⁹, R¹⁰ and R¹¹ are as previously defined), hydroxyethyl and oligomers and alkyl ethers thereof, aryloxy, substituted aryloxy, heteroaryloxy, substituted heteroaryloxy, heterocycloalkyloxy, substituted heterocycloalkyloxy, thioalkyl, carbonyl, thiocarbonyl, benzyl, carboxamido, sulfonamido, aryl, substituted aryl, heteroaryl, substituted heteroaryl, heterocycloalkyl and substituted heterocycloalkyl, or when taken together form a 5, 6 or 7 member carbocyclic ring or a heterocyclic ring containing oxygen, sulfur or nitrogen;
R¹⁴, and R¹⁵ are each independently selected from the group consisting of hydrogen, halogen, cyano, (C₁₋₆)alkyl, (C₃₋₇)cycloalkyl, trifluoromethyl, amino, alkylamino, dialkylamino, hydroxy, nitro, COOH, COOR⁹, CONHR¹⁰, CONR¹¹ (where R⁹, R¹⁰ and R¹¹ are as previously defined), hydroxyethyl and oligomers and alkyl ethers thereof, aryloxy, substituted aryloxy, heteroaryloxy, substituted heteroaryloxy, heterocycloalkyloxy, substituted heterocycloalkyloxy, thioalkyl, carbonyl, thiocarbonyl, benzyl, carboxamido, sulfonamido, aryl, substituted aryl, heteroaryl, substituted heteroaryl, heterocycloalkyl and substituted heterocycloalkyl, or when taken together form a 5, 6 or 7 member carbocyclic ring or a heterocyclic ring containing oxygen, sulfur or nitrogen;
X and Y are each independently divalent linking moieties selected from the group consisting of (C₁₋₆)alkyl, (C₃₋₇)cycloalkyl, heterocyclic, substituted heterocyclic, heterocycloalkyl, substituted heterocycloalkyl aryl and substituted aryl; and
m and n are each independently 0 or 1,
with the proviso that the bifunctional molecule does not have the following structure:

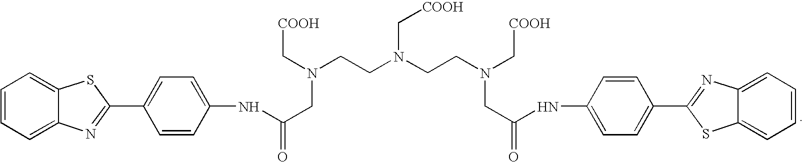

9. The contrast imaging agent of claim 8, wherein X and Y are independently phenyl or substituted phenyl, and wherein the phenyl or substituted phenyl carries the group: 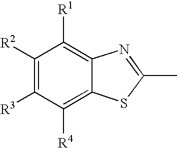
at the ortho, meta or para position.

10. The contrast imaging agent of claim 7, wherein the metal entity is a paramagnetic metal ion selected from the group consisting of gadolinium III (Gd³⁺), chromium III (Cr³⁺), dysprosium III (Dy³⁺), iron III (Fe³⁺), manganese II (Mn²⁺), and ytterbium III (Yb³⁺).

11. The contrast imaging agent of claim 7, wherein the metal entity is a radionuclide selected from the group consisting of technetium-99m (^{99m}Tc), gallium-67 (⁶⁷Ga), yttrium-91 (⁹⁰Y), indium-111 (¹¹¹In), rhenium-186 (¹⁸⁶Re), and thallium-201 (²⁰¹Tl).

12. The contrast imaging agent of claim 7, wherein the metal entity is a stable paramagnetic isotope selected from the group consisting of carbone-13 (¹³C) or fluorine-19 (¹⁹F).

13. A pharmaceutical composition comprising an effective amount of at least one bifunctional molecule of claim 1 or 2, or a physiologically tolerable salt thereof, and at least one pharmaceutically acceptable carrier.

14. A pharmaceutical composition comprising an effective amount of at least one bifunctional molecule of claim 4 or 6, or a physiologically tolerable salt thereof, and at least one pharmaceutically acceptable carrier.

15. A pharmaceutical composition comprising an imaging effective amount of at least one contrast imaging agent of claim 7 or 8, or a physiologically tolerable salt thereof, and at least one pharmaceutically acceptable carrier.

16. A method for reducing or inhibiting amyloid toxicity in a system, comprising contacting the system with a bifunctional molecule of claim 1 or 4, or a pharmaceutical composition thereof.

17. The method of claim 16, wherein the cell, biological fluid, or biological tissue originates from a patient suspected of having a pathophysiological condition associated with amyloid accumulation.

18. The method of claim 17, wherein the pathophysiological condition is associated with accumulation of the amyloid-β peptide.

19. A method for treating a patient with a pathophysiological condition associated with amyloid accumulation, comprising administering to the patient an effective amount of a bifunctional molecule of claim 1 or 4, or a pharmaceutical composition thereof.

20. The method of claim 19, wherein the pathophysiological condition is associated with accumulation of the amyloid-β peptide.

21. The method of claim 20, wherein the pathophysiological condition is selected from the group consisting of Alzheimer's disease, Down's syndrome, Lewy body dementia, hereditary cerebral hemorrhage with amyloidosis (Dutch type), Guam Parkinson-Dementia, and head trauma.

22. A method for detecting the presence of amyloid deposits in a system comprising steps of:
contacting the system with an imaging effective amount of a contrast imaging agent of claim 7, or a pharmaceutical composition thereof, under conditions to allow the contrast imaging agent to interact with any amyloid deposit present so that the interaction results in binding of the contrast imaging agent to the amyloid deposit;
detecting any amyloid deposit present in the system and bound to the contrast imaging agent, using an imaging technique; and
generating one or more images of at least part of the system.

23. The method of claim 22, wherein the amyloid deposits present in the system are formed by accumulation of the amyloid-β peptide.

24. The method of claim 23, wherein the system is selected from the group consisting of a cell, a biological fluid, and a biological tissue.

25. The method of claim 24, wherein the cell, biological fluid, or biological tissue originates from a patient suspected of having a pathophysiological condition associated with amyloid accumulation, or from a patient receiving a treatment for a pathophysiological condition associated with amyloid accumulation.

26. The method of claim 24, wherein the cell, biological fluid, or biological tissue has been contacted with a potential therapeutic agent for the treatment of a pathophysiological condition associated with amyloid accumulation.

27. The method of claim 26, wherein said method is used to identify potential therapeutic agents for the treatment of a pathophysiological condition associated with amyloid accumulation.

28. The method of claim 24, wherein said method is used to diagnose a pathophysiological condition associated with amyloid accumulation, to follow the progression of a pathophysiological condition associated with amyloid accumulation, or to monitor the response of a patient to a treatment for a pathophysiological condition associated with amyloid accumulation.

29. A method for detecting the presence of amyloid deposits in a patient comprising steps of:
administering to the patient an imaging effective amount of a contrast imaging agent of claim 7, or a pharmaceutical composition thereof, under conditions to allow the contrast imaging agent to interact with any amyloid deposit present so that the interaction results in binding of the contrast imaging agent to the amyloid deposit;
detecting any amyloid deposit present in the patient and bound to the contrast imaging agent, using an imaging technique; and
generating one or more images of at least part of the body of the patient.

30. The method of claim 29, wherein said method is used to localize amyloid deposits in a patient.

31. The method of claim 29, wherein said method is used to diagnose a pathophysiological condition associated with amyloid accumulation, to follow the progression of a pathophysiological condition associated with amyloid accumulation, or to monitor the response of a patient to a treatment for a pathophysiological condition associated with amyloid accumulation.

32. The method of claim 22 or 29, wherein the metal entity is a paramagnetic metal ion selected from the group consisting of gadolinium III (Gd³⁺), chromium III (Cr³⁺), dysprosium III (Dy³⁺), iron III (Fe³⁺), manganese II (Mn²⁺), and ytterbium III (Yb³⁺), and wherein the detection is carried out by Magnetic Resonance Imaging (MRI); and MR images are generated.

33. The method of claim 22 or 29, wherein the metal entity is a radionuclide selected from the group consisting of technetium-99m (^{99m}Tc), gallium-67 (⁶⁷Ga), yttrium-91 (⁹⁰Y), indium-111 (¹¹¹In), rhenium-186 (¹⁸⁶Re), and thallium-201 (²⁰¹Tl), and wherein the detection is carried out by Single Photon Emission Computed Tomography (SPECT) and SPECT images are generated.
